(19) United States
(12) Patent Application Publication
(10) Pub. No.: US 2026/0231297 A1
(43) Pub. Date: Aug. 6, 2026

(54) PORTABLE HAND WARMER STORAGE BOX AND HAND WARMER ASSEMBLY

(71) Applicant: Guangdong Aoyun Technology Co., Ltd., Huizhou (CN)

(72) Inventor: Xueping Zhu, Huizhou (CN)

(21) Appl. No.: 19/634,353

(22) Filed: Mar. 31, 2026

Related U.S. Application Data
(63) Continuation-in-part of application No. 19/279,069, filed on Jul. 24, 2025, now Pat. No. 12,616,607.
Application No. 19/279,069 is a continuation-in-part of application No. 19/089,189, filed on Mar. 25, 2025, now Pat. No. 12,451,710.

(30) Foreign Application Priority Data
Oct. 20, 2023 (CN) 202322833425.8
Jul. 9, 2024 (CN) 202421622634.6
Mar. 19, 2025 (CN) 202520496218.4
Jul. 15, 2025 (CN) 202521483945.3

(51) Int. Cl. H05B 3/06

(52) U.S. Cl. CPC H05B 3/06

(57) ABSTRACT

A portable hand warmer assembly includes a case body with at least one receiving portion for receiving at least one electronic hand warmer, and the case body includes a first charging unit, after the at least one electronic hand warmer is received in the at least one receiving portion, the at least one electronic hand warmer is charged by the first charging unit.

### CROSS-REFERENCE TO RELATED APPLICATIONS

The application is a Continuation-in-Part of the U.S. application Ser. No. 19279069, filed on July 24, 2025, and entitled "PORTABLE HAND WARMER CHARGING CASE AND HAND WARMER ASSEMBLY", now pending, which is a Continuation-in-Part of US 19089189 filed on March 25, 2025, now patented; the entire disclosures of which are incorporated herein by reference.

### TECHNICAL FIELD

The present disclosure relates to the technical field of hand warmers, and in particular, to a portable hand warmer storage box and a hand warmer assembly.

### BACKGROUND

Whether it is double-sided or single-sided, existing hand warmers on the market are usually only able to warm one hand or one body part, making it difficult to simultaneously warm both hands or multiple body parts. Especially when a user needs to perform different actions with the hands separately or when a user intends to use the hand warmer to warm multiple body parts, it is hard for the current hand warmers to meet the need of the user. Furthermore, to charge an existing hand warmer, the existing hand warmer needs to be connected to an external power supply through a power cable. However, when people go out, especially at outdoors, it is difficult to store the hand warmers, let alone power supply the hand warms. Therefore, there is an urgent need on the market for a storage box that is convenient to carry or charge a hand warmer.

### SUMMARY

The present disclosure aims to provide a portable hand warmer storage box and a hand warmer assembly. The problem of inconvenience of charging of the existing hand warmer at outdoors is solved.

In order to solve the technical problem, the technical scheme provided by the present disclosure is as follows.

A portable hand warmer assembly includes two electronic hand warmers and a storage box. Each of the electronic hand warmers provided with a heating element and having an upper surface for heating and a lower surface opposite to the upper surface; wherein the electronic hand warmers are detachably connected to each other in a manner that each of the electronic hand warmers is capable of warming individually through the upper surface, or the two electronic hand warmers are stacked by stacking the lower surfaces together to form a combined unit for warming. The storage box includes a case body with at least one receiving chamber for receiving the two electronic hand warmers, wherein the case body includes an opening corresponding to the at least one receiving chamber, allowing the two electronic hand warmers to be inserted into the at least one receiving chamber through the opening individually or in the combined unit.

A portable hand warmer assembly includes at least two electronic hand warmers and a storage box. The storage box includes a case body with at least one receiving chamber for receiving the at least two electronic hand warmers, and an openable cover covering the case body.

A storage box includes a case body with at least one receiving portion for receiving the at least one electronic hand warmer, and the case body includes a first charging unit, after the at least one hand warmer is received in the at least one receiving portion, the at least one hand warmer is charged by the first charging unit.

Beneficial effects of the present disclosure are as follows: The present invention breaks the limitations of a single hand warmer by configuring at least two detachable electronic hand warmers. Users can separate the electronic hand warmers and use their respective upper surfaces to generate heat separately, easily meeting the needs of heating their hands separately or sharing with others. Their lower surfaces can be adhered to each other to form a combined unit, which can make the upper surfaces of the two electronic hand warmers jointly heat up to the outside, thereby further expanding the heating area and improving the overall heating effect; by equipping a storage box with a receiving chamber and an opening, multiple electronic hand warmers can be uniformly and orderly placed in the warehouse for centralized storage, effectively solving the problem of multiple electronic hand warmers being easily scattered and lost when carried.

The storage box not only includes a receiving chamber for placing the electronic hand warmer unit, but also has a cover that can cover the receiving chamber. When idle or carried out, the cover covers the storage compartment, effectively preventing the intrusion of external dust and the scratching of hard objects during daily carrying. It also prevents damage caused by falling during carrying, thereby improving the protection and portability of the storage box. The receiving chamber can be a flat or a concave receiving portion, and the storage box can further integrates a charging unit, when the battery level of the hand warmer is low, users only need to place it in the receiving portion and directly charge it through the charging unit; the storage box combines the function of a charging compartment, freeing traditional hand warmers from reliance on external power cords and fixed power sources outdoors, achieving the convenience of "storage and charging", and significantly improving the user experience.

### BRIEF DESCRIPTION OF THE DRAWINGS

In order to explain the technical solutions of the embodiments of the present disclosure more clearly, the following will briefly introduce the accompanying drawings used in the embodiments. Apparently, the drawings in the following description are only some embodiments of the present disclosure. Those of ordinary skill in the art can obtain other drawings based on these drawings without creative work.

FIG. 1 is a schematic diagram of a charging case of the present disclosure;

FIG. 2 is a schematic diagram of a charging case in another viewing angle according to the present disclosure;

FIG. 3 is a schematic state diagram of a hand warmer assembly according to the present disclosure;

FIG. 4 is a partially exploded diagram of a hand warmer assembly according to the present disclosure;

FIG. 5 is an exploded diagram of a main body according to the present disclosure;

FIG. 6 is a schematic diagram of assembling a first inner shell and a first circuit board according to the present disclosure;

FIG. 7 is a structural diagram of a light guide hood according to the present disclosure;

FIG. 8 is an exploded diagram of a cover body according to the present disclosure;

FIG. 9 is a three-dimensional diagram of a hand warmer according to the present disclosure;

FIG. 10 is an exploded view of a hand warmer according to the present disclosure;

FIG. 11 is a schematic diagram of integrally assembling a first hand warmer and a second hand warmer according to the present disclosure;

FIG. 12 is an exploded diagram of a first hand warmer and a second hand warmer according to the present disclosure;

FIG. 13 is a schematic diagram of a hand warmer module of another embodiment of the present disclosure according to another embodiment;

FIG. 14 is a schematic diagram of a charging case of another embodiment of the present disclosure;

FIG. 15 is a schematic diagram of a hand warmer of another embodiment of the present disclosure;

FIG. 16 is a schematic diagram of a portable hand warmer charging case and hand warmer in charging state according to another embodiment of the present disclosure;

FIG. 17 shows the hand warmer taken out of the charging case of FIG. 16;

FIG. 18 is an exploded view of FIG. 17;

FIG. 19 is a schematic diagram of a portable hand warmer charging case and hand warmer in charging state according to further another embodiment of the present disclosure;

FIG. 20 shows the hand warmer taken out of the charging case of FIG. 19;

FIG. 21 is an exploded view of FIG. 20;

FIG. 22 is a schematic diagram of a portable hand warmer charging case and hand warmer in charging state according to further another embodiment of the present disclosure;

FIG. 23 shows the hand warmer taken out of the charging case of FIG. 22;

FIG. 24 is an exploded view of FIG. 23;

FIG. 25 is a schematic diagram of a portable hand warmer charging case and hand warmer in charging state according to further another embodiment of the present disclosure;

FIG. 26 shows the hand warmer taken out of the charging case of FIG. 25;

FIG. 27 is an exploded view of FIG. 26;

FIG. 28 is a schematic diagram of a portable hand warmer charging case and hand warmer in charging state according to further another embodiment of the present disclosure;

FIG. 29 is an exploded view of FIG. 28;

FIG. 30 is a schematic diagram of a portable hand warmer charging case and hand warmer in charging state according to further another embodiment of the present disclosure;

FIG. 31 is an exploded view of FIG. 30;

FIG. 32 shows two hand warmers are stacked are stacked by stacking the lower surfaces together to form a combined unit;

FIG. 33 is a schematic view of a storage box and two hand warmers;

FIG. 34 is another view of the storage box and the two hand warmers of FIG. 33.

### DETAILED DESCRIPTION OF THE EMBODIMENTS

The accompanying drawings in the embodiment of the present disclosure are combined. The technical scheme in the embodiment of the present disclosure is clearly and completely described. Obviously, the described embodiment is only a part of the embodiment of the present disclosure, but not all embodiments are based on the embodiment of the present disclosure, and all other embodiments obtained by ordinary technicians in the field on the premise of not doing creative work belong to the protection range of the present disclosure.

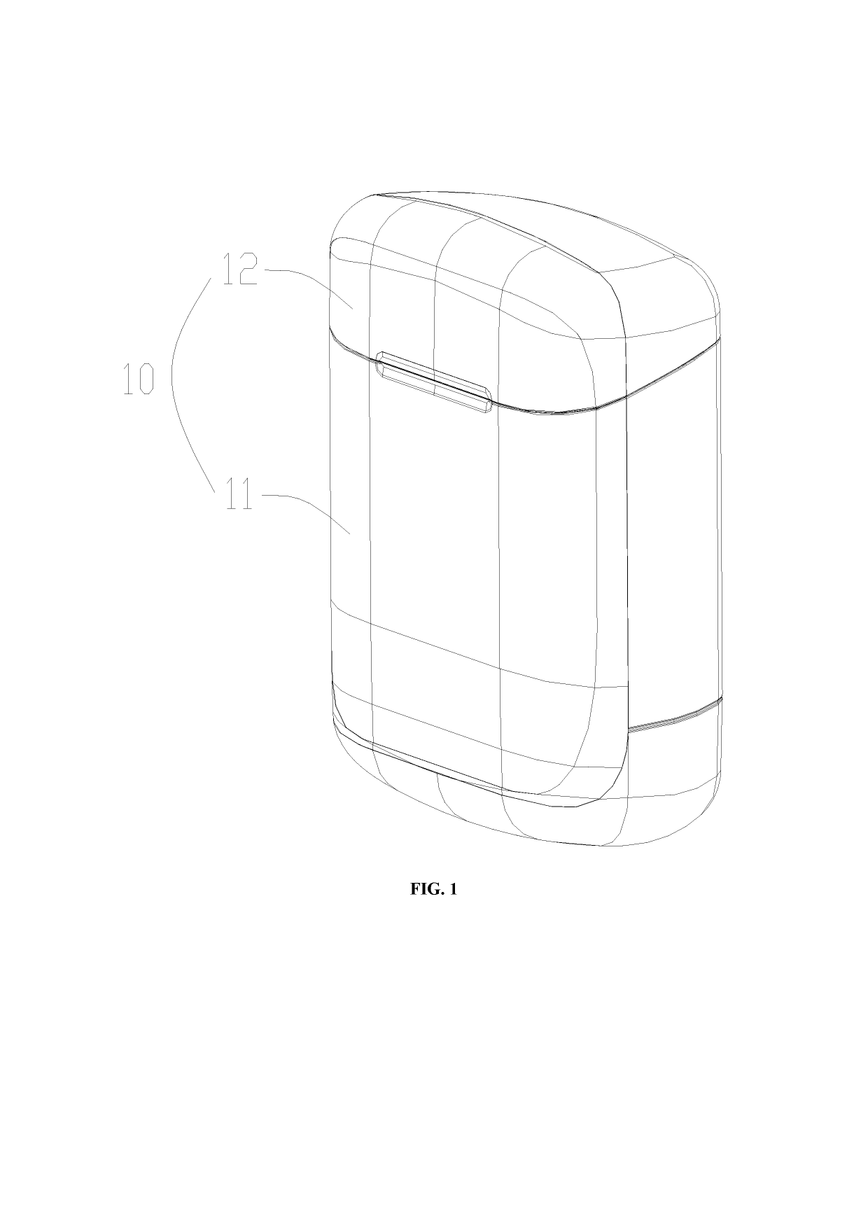

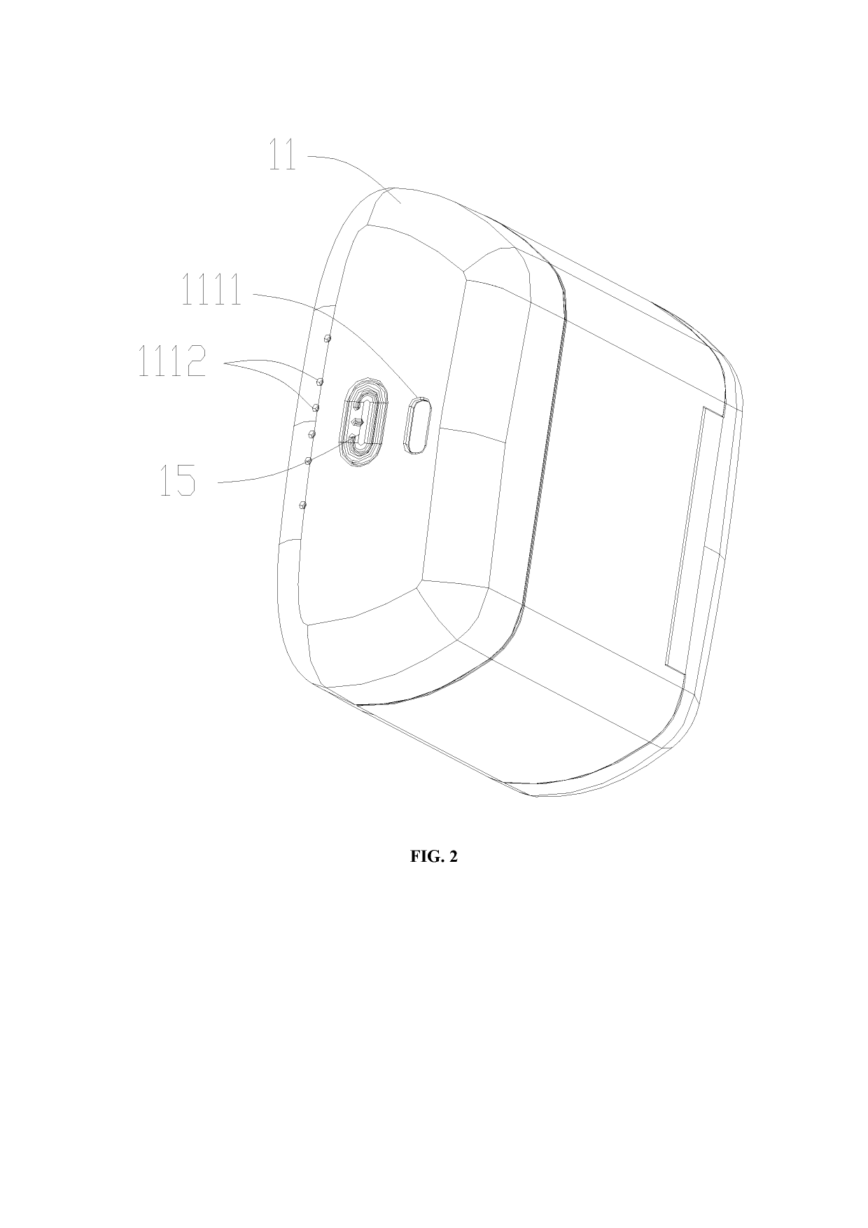

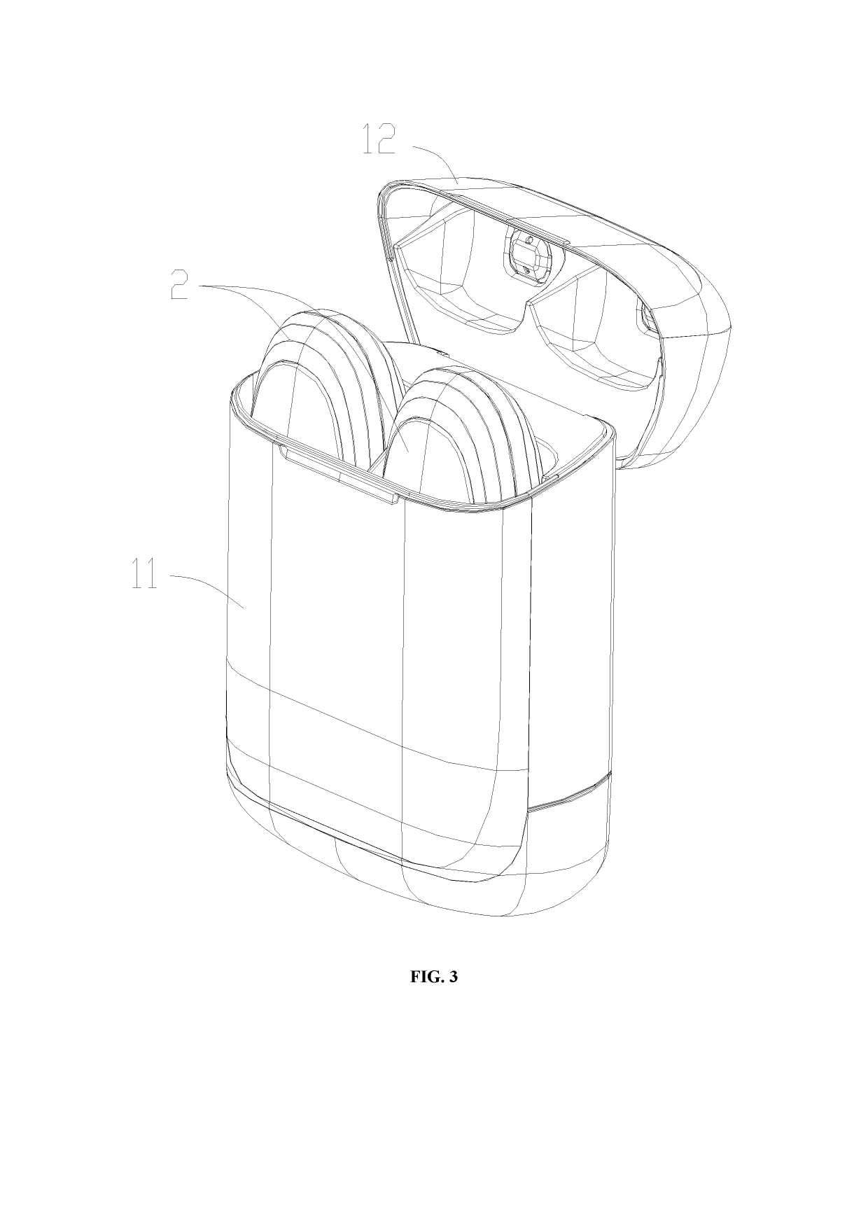

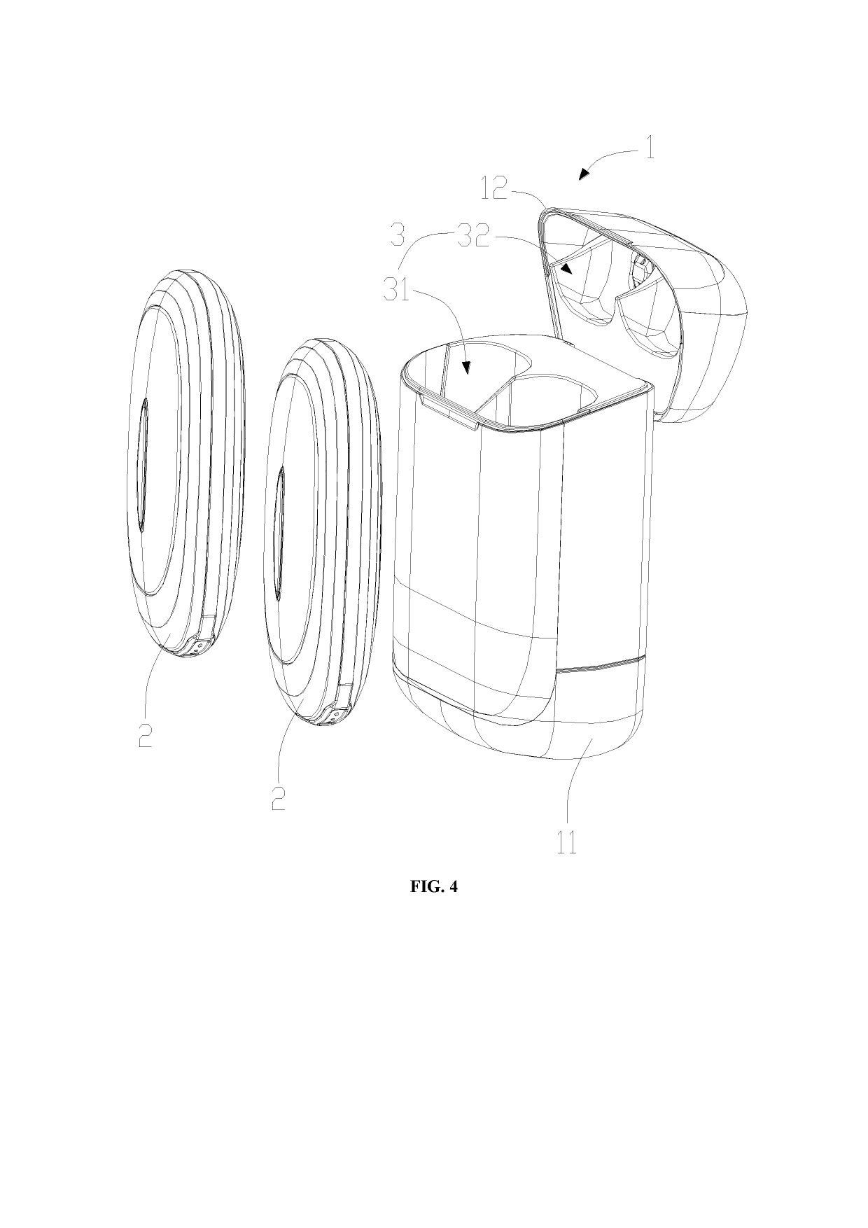

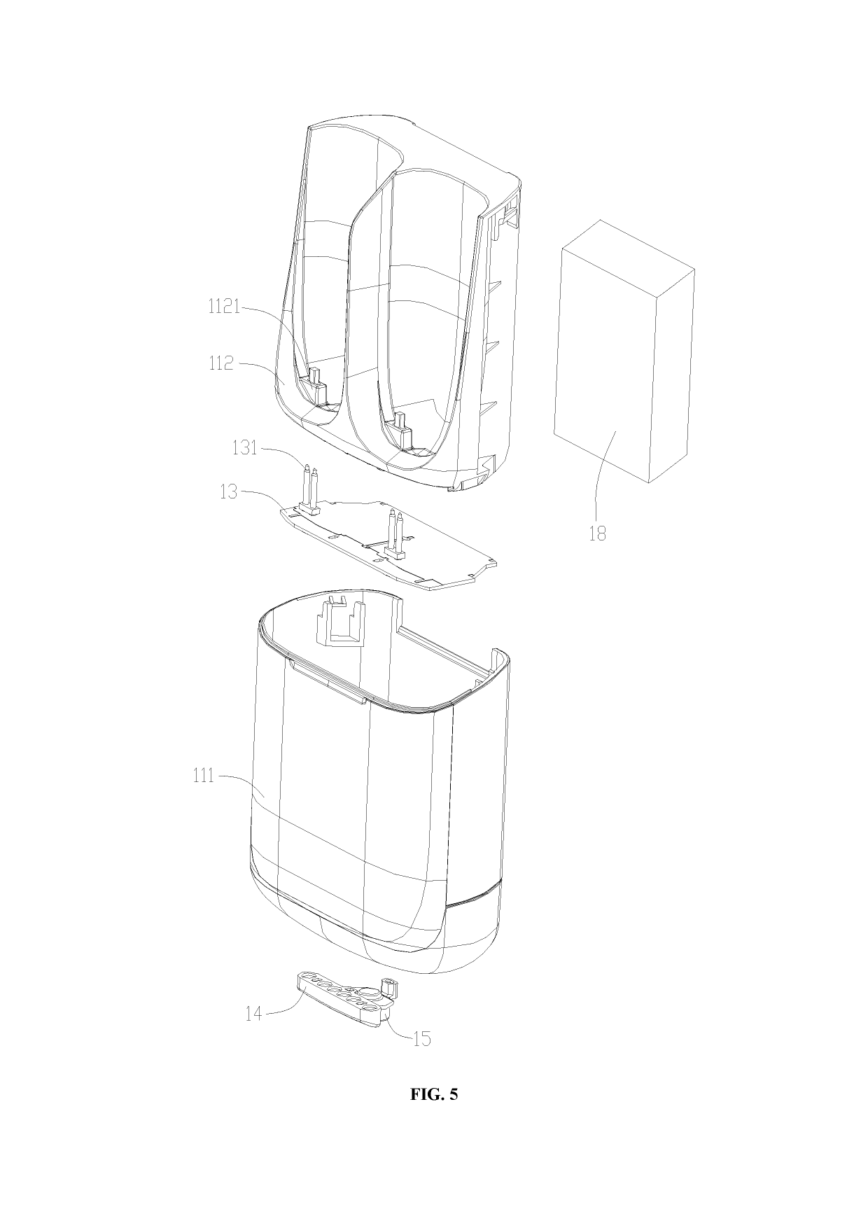

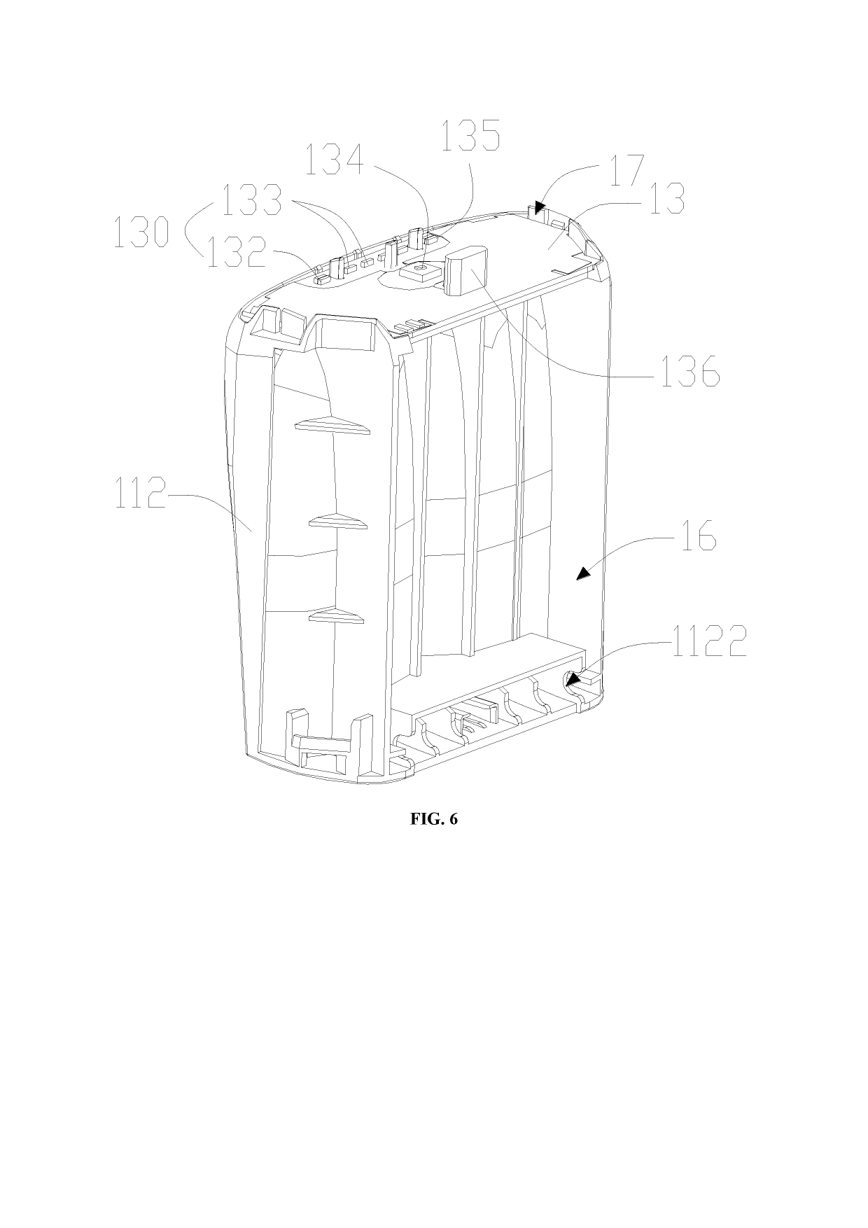

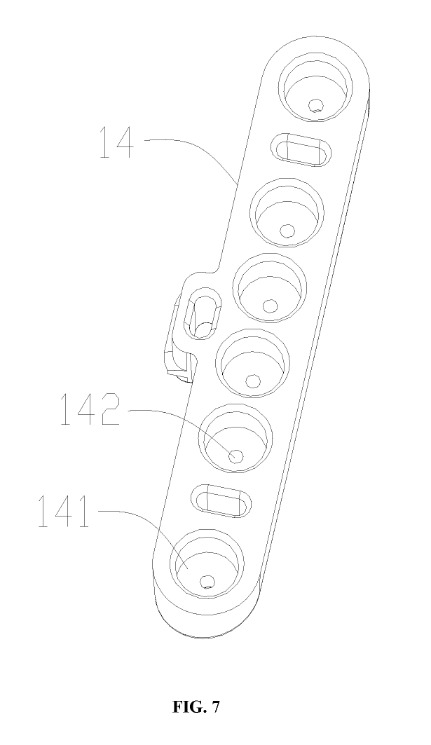

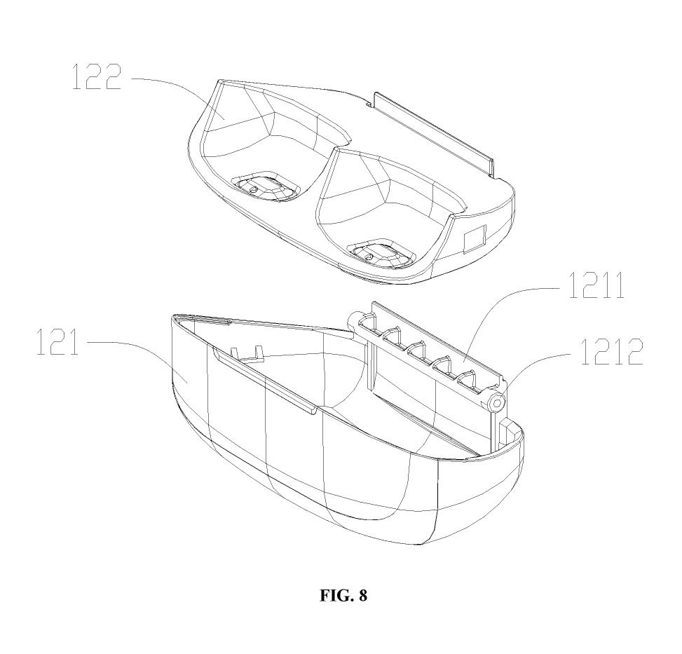

Referring to FIG. 1 to FIG. 8, a portable hand warmer charging case 1 in the embodiments of the present disclosure is provided.

The portable hand warmer charging case 1 includes a case body 10. A receiving chamber 3 configured to place a hand warmer 2 for charging is provided on the case body 10. The case body 10 is provided with a first circuit board 13, a first charging unit 131, and a first battery 18; the first battery 18 and the first charging unit 131 are electrically connected to the first circuit board 13; when the hand warmer 2 is placed in the receiving chamber 3, the first circuit board 13 is configured to output electrical energy, which is output by the first battery 18, from the first charging unit 131; and the first charging unit 131 is configured to be adapted to a second charging unit 24 on the hand warmer 2 for use and transmit the electrical energy output by the first circuit board 13 to the second charging unit 24 on the hand warmer 2.

In this embodiment, the receiving chamber 3 configured to accommodate the hand warmer 2 is provided on the charging case 1, and the first charging unit 131 configured to supply power to the hand warmer 2 is configured on the charging case 1. In this way, when the hand warmer 2 is stored and placed in the receiving chamber 3, the first charging unit 131 can be close to or in contact with the hand warmer 2 to enable the first charging unit 131 to transmit the electrical energy output by the first circuit board 13 from the first battery 18 to the second charging unit 24, so as to charge the hand warmer 2. If a user carries the charging case 1 at outdoors, the user can charge the hand warmer 2 when the hand warmer 2 runs out of power. The problem of inconvenience of charging of the existing hand warmer 2 at outdoors is solved.

In an embodiment, since the hand warmer 2 is designed to be used for the hands of a user, most hand warmers 2 are generally flat. The thickness of the receiving chamber 3 is adapted to the thickness of the hand warmer 2 to make the overall appearance of the charging case 1 harmonious. The first circuit board 13 is located at the bottom of the receiving chamber 3, and the first battery 18 is located behind the case body 10. Of course, in other embodiments, the first circuit board 13 and the first battery 18 may be both located at the bottom, back, or front side of the receiving chamber 3. Or, the first circuit board 13 may be located in front of the receiving chamber 3, and the first battery 18 may be located at the back of the receiving chamber 3.

In an embodiment, the first circuit board 13 is provided with a battery level indicator lamp 133 configured to indicate a battery level state of the first battery 18 and/or a charging state of the hand warmer 2, so that a user can learn the battery level of the first battery 18 and whether the hand warmer 2 is fully charged.

In an embodiment, the indicator lamp unit 130 includes a plurality of light-emitting bodies 135 arranged on the first circuit board 13, and the plurality of light-emitting bodies 135 are arranged side by side. Of course, in other embodiments, the plurality of light-emitting bodies 135 can alternatively be arranged in a surrounding manner, irregularly arranged, distributed in an arc shape, or the like. Therefore, if the first battery 18 only has a half battery level, the first circuit board 13 can control half of the light-emitting bodies 135 to emit light. If the first battery 18 has full battery level, the first circuit board 13 can control all the light-emitting bodies 135 to emit light, so that a user can learn the battery level of the first battery 18. Of course, the color of light emitted by the light-emitting bodies 135 can alternatively be used to indicate the battery level of the first battery 18. For example, if the first battery 18 has full battery level, the light-emitting bodies 135 emit green light; and if the first battery 18 has low battery, the light-emitting bodies 135 emit red light. The light-emitting bodies 135 can be light-emitting diode (LED) lamps, LEDs, or the like.

In an embodiment, the case body 10 is further provided with a light guide hood 14; the light guide hood 14 is provided with an accommodating slot 141 at a position corresponding to each light-emitting body 135; the light-emitting body 135 is located in the accommodating slot 141; a first light guide hole 142 that is communicated with the accommodating slots 141 is provided on the light guide hood 14; and a second light guide hole 1112 is provided at a position, corresponding to the first light guide hole 142, on the case body 10. Specifically, the light guide hood 14 is made of an opaque material, which means that the light from the light-emitting bodies 135 can only pass through the first light guide hole 142 and then exit from the second light guide hole 1112, which prevents the problem of poor indication effect caused by the mutual impact of the light between the plurality of light-emitting bodies 135.

In an embodiment, the indicator lamp unit 130 includes a battery level indicator lamp 133 and a charging state indicator lamp 132, and the charging state indicator lamp 132 is located next to the battery level indicator lamp 133. The battery level indicator lamp 133 can indicate a battery level of the first battery 18 according to the number of the light-emitting bodies that are lit up, and the charging state indicator lamp 132 indicates a charging state of the hand warmer 2 according to the color of the emitted light, to facilitate use.

In an embodiment, the first circuit board 13 is provided with a function button 134; and the case body 10 is provided with a pressing member 15 connected to the function button 134. When the hand warmer 2 is placed in the receiving chamber 3, a user can press the function button 134 by pressing the pressing member 15. The first circuit board 13 detects an electrical signal of the function button 134 and outputs the electrical energy of the first battery 18 from the first charging unit 131 to control the charging of the hand warmer 2. Of course, by pressing the function button 134, the first circuit board 13 can further drive the indicator lamp to be turned on, so that a user can learn the remaining power of the first battery 18.

In the above embodiments, a first charging interface 136 is provided on the first circuit board 13; and a first avoidance hole 1111 configured to avoid the first charging interface 136 is provided on the case body 10. The first charging interface 136 is configured to be externally connected to a power supply. After the power supply is connected, the first battery 18 can be charged via the first circuit board 13, so that a user can charge the first battery 18 if the charging case 1 has low battery. Of course, in other embodiments, the charging case 1 can further use wireless charging to charge the first battery 18.

In an embodiment, for the structural compactness and beauty, the indicator lamp unit 130, the function button 134, and first charging interface 136 are arranged side by side in a thickness direction of the charging case 1; and the function button 134 is located between the indicator lamp unit 130 and the first charging interface 136.

In the above embodiment, the case body 10 includes a main body 11 and a cover body 12 connected to the main body 11 to form the receiving chamber 3; and the first circuit board 13, the first battery 18, the pressing member 15, the first avoidance hole 1111, and the second light guide hole 1112 are all located on the main body 11. The cover body 12 is used to cooperate with the main body 11 to form the closed receiving chamber 3. When the hand warmer 2 is placed on the placement slot, the hand warmer 2 can be completely wrapped to achieve an effect of protecting the hand warmer 2, so that the hand warmer is convenient to carry when people go out. Of course, in other embodiments, the cover body 12 may not be needed, and the structure of the case body 10 is not limited here.

For the structural compactness and ease of production and manufacturing of the product, the receiving chamber 3 includes a first placement slot 31; the first charging unit 131 is a conductive probe; the conductive probe is arranged at a bottom of the first placement slot 31; the main body 11 includes a first outer cover 111 and a first inner shell 112 connected to the first outer cover 111 to form the first placement slot 31, a first mounting chamber 17, and a second mounting chamber 16; the first inner shell 112 is located on an inner side of the first outer cover 111; the first mounting chamber 17 is located below the first placement slot 31; and the first circuit board 13 and the light guide hood 14 are arranged in the first mounting chamber 17. The first battery 18 is arranged in the second mounting chamber 16, and the second mounting chamber 16 is located behind the first placement slot 31. The pressing member 15, the first avoidance hole 1111, and the second light guide hole 1112 are all located on the first outer cover 111.

In an embodiment, the cover body 12 is rotatably connected to the main body 11. Of course, in other embodiments, the cover body 12 and the main body 11 may be further connected to each other through magnetic suction, a buckle, or the like.

In an embodiment, the receiving chamber 3 includes a second placement slot 32; the cover body 12 includes a second outer cover 121 and a second inner shell 122 connected to the second outer cover 121 to form the second placement slot 32; the second inner shell 122 is at least partially located on an inner side of the second outer cover 121; a bottom of the second outer cover 121 extends downwards to form a connecting panel 1211; two sides of the connecting panel 1211 protrude to form a rotating shaft portion 1212; The first outer shell 111 is connected to the first inner shell 112 to form a limiting rotating hole 1122. The rotating shaft portion 1212 is arranged in the limiting rotating hole 1122 to achieve rotatable connection between the cover body 12 and the main body 11. Furthermore, one side of the cover body 12 away from the rotating shaft portion 1212 can be stably connected to the main body 11 through magnetic suction, a buckle, or the like, so as to prevent the cover body 12 from being automatically opened.

Based on the above charging case 1, this embodiment further provides a hand warmer assembly.

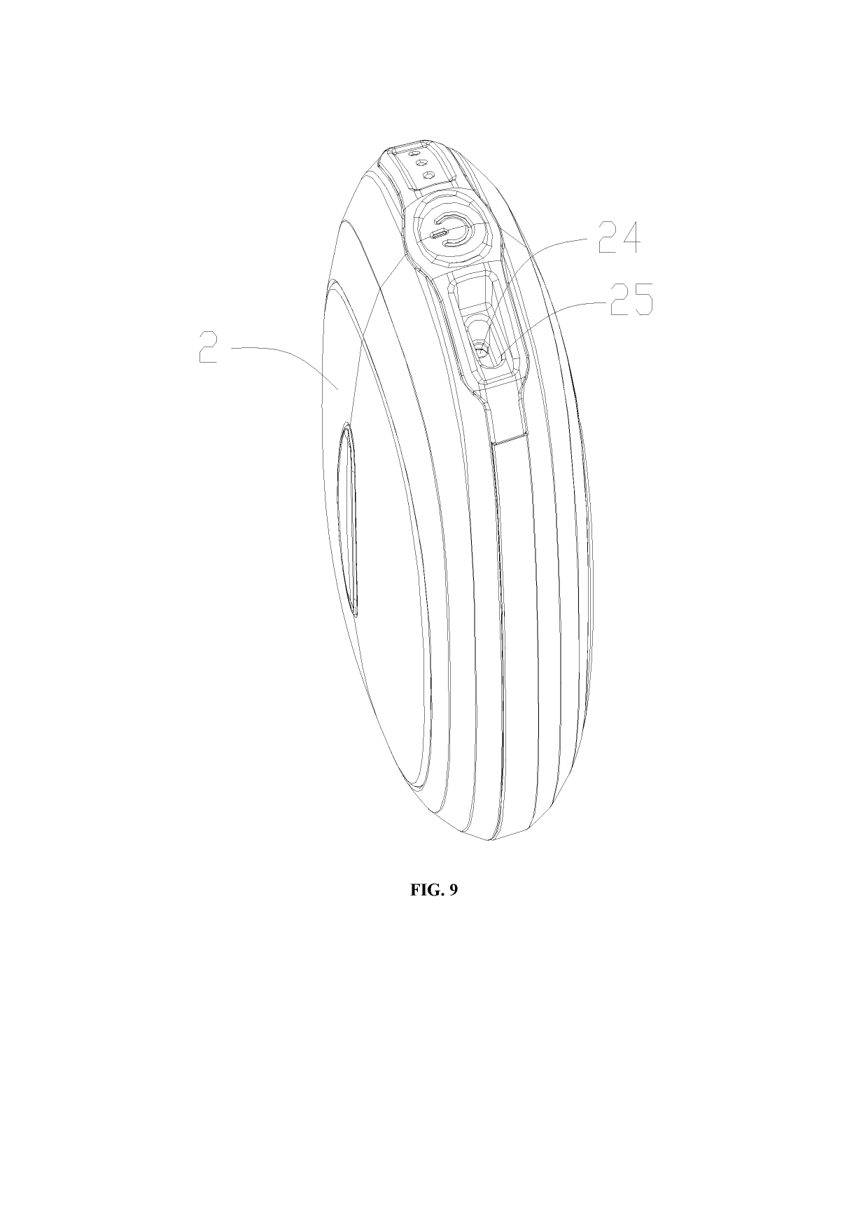

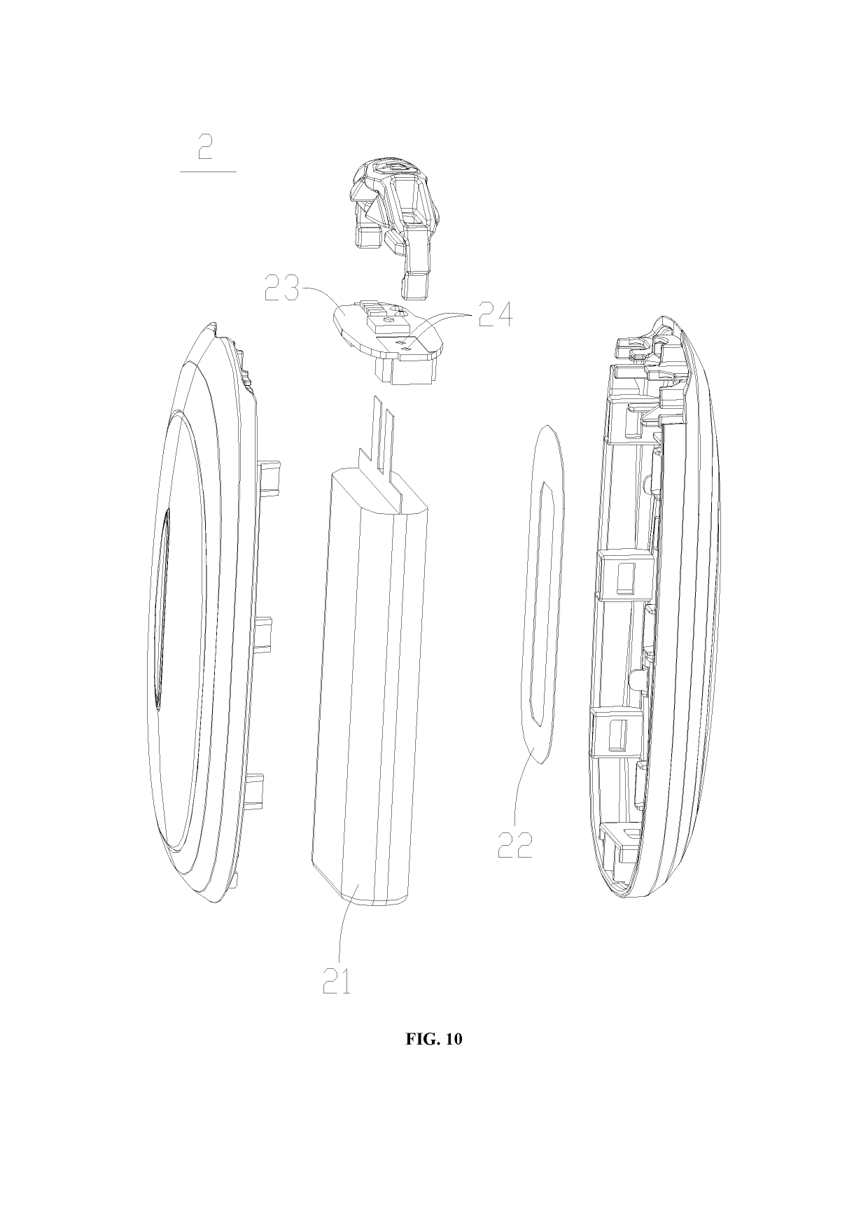

Referring to FIG. 1 to FIG. 10, the hand warmer assembly includes at least one hand warmer 2 and the charging case 1 as described above. The hand warmer 2 is provided with a second circuit board 23, a heating element 22, a second battery 21, and a second charging unit 24; and the heating element 22, the second battery 21, and the second charging unit 24 are all electrically connected to the second circuit board 23. When the hand warmer 2 is placed in the receiving chamber 3, the second charging unit 24 is close to or in contact with the first charging unit 131; the first charging unit 131 is configured to output the electrical energy, which is output by the first circuit board 13, to the second charging unit 24; and the second charging unit 24 charges, via the second circuit board 23, the second battery 21 with the electrical energy output by the first charging unit 131. By the use of the charging case 1, it is convenient for a user to charge the hand warmer 2 when the user goes out.

In an embodiment, the first charging unit 131 is a conductive probe, and the conductive probe is arranged at a bottom of the receiving chamber 3. The second charging unit 24 is a charging contact. The charging contact is located at a bottom of the hand warmer 2. When the hand warmer 2 is placed in the receiving chamber 3, the charging contact is in contact with the conductive probe to allow the electrical energy output by the first battery 18 to charge the second battery 21 through the first circuit board 13, the conductive probe, the charging contact, and second circuit board 23.

In an embodiment, a position, corresponding to the charging contact, on the hand warmer 2 is recessed inwards to form a groove 25; an embedded block 1121 is arranged at a position, corresponding to the groove 25, on the receiving chamber 3; and the embedded block 1121 is arranged in the groove 25 when the hand warmer 2 is placed in the receiving chamber 3.

In an embodiment, the case body 10 and the hand warmer 2 can be further fixed through interference fit, magnetic suction, and buckle connection, which can improve the stability of contact between the conductive probe and the charging contact too.

In other embodiments, both the first charging unit 131 and the second charging unit 24 can be coils. Namely, when the hand warmer 2 is placed in the receiving chamber 3, the first charging unit 131 and the second charging unit 24 are close to and interact with each other, so that the electrical energy output by the first battery 18 charges the second battery 21 through the first circuit board 13, the conductive probe, the charging contact, and the second circuit board 23, achieving wireless charging of the hand warmer 2. Or, the first charging unit 131 and the second charging unit 24 can be a male interface and a female interface adapted for use, and can charge the hand warmer 2 when the hand warmer 2 is placed in the receiving chamber 3.

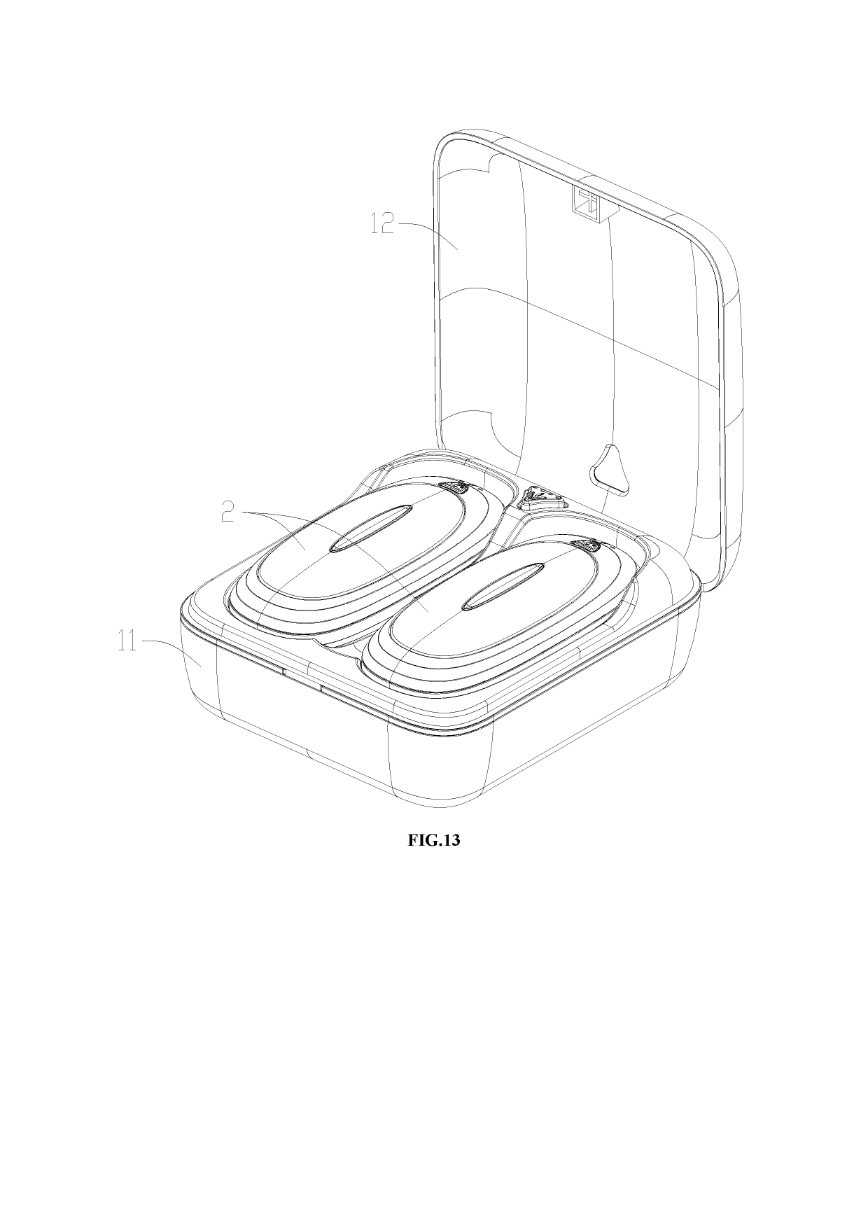

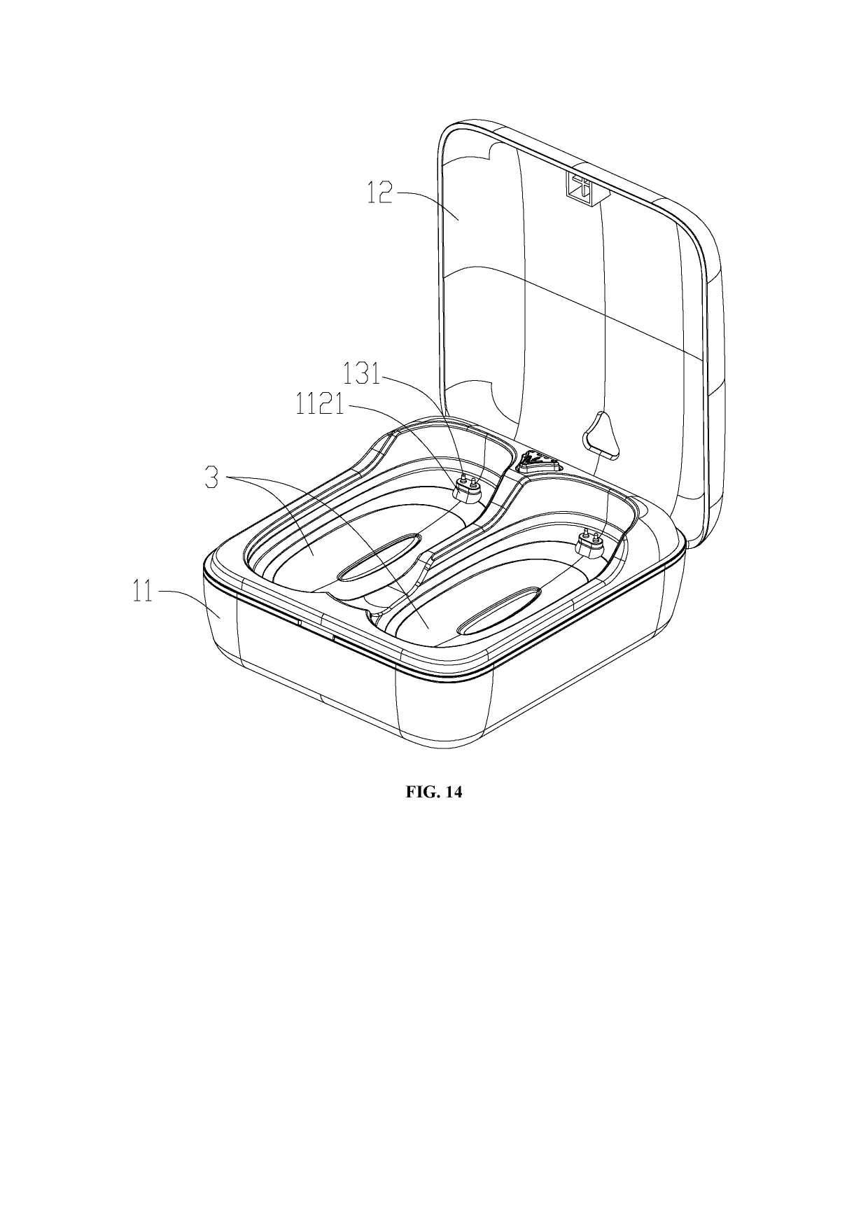

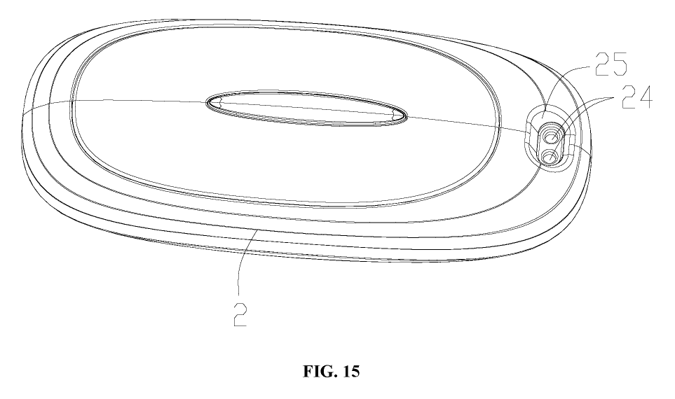

In an embodiment, the charging contact can be arranged at the bottom of the hand warmer. Namely, the hand warmer 2 is inserted vertically into the receiving chamber 3, namely, into the receiving chamber 3 in a height direction of the hand warmer 2. In other embodiments, the charging contact can be arranged on a side wall of the hand warmer 2. Namely, the hand warmer 2 can be inserted into the receiving chamber 3 in a width direction of the hand warmer 2. In other embodiments, the charging contact can alternatively be arranged on an upper or lower surface of the hand warmer, so that the hand warmer 2 can be placed in the receiving chamber 3 in a flat state. Referring to FIG.13 to FIG. 15, that is, the hand warmer 2 can be placed in the receiving chamber 3 in a thickness direction of the hand warmer 2. The manufacturers can customize the configuration according to their needs.

In an embodiment, there is one receiving chamber 3, which means that the charging case 1 is only suitable for charging one hand warmer 2.

In an embodiment, there are a plurality of receiving chambers 3. Each receiving chamber 3 is provided with one first charging unit 131, which means that the charging case 1 can simultaneously charge a plurality of hand warmer 2.

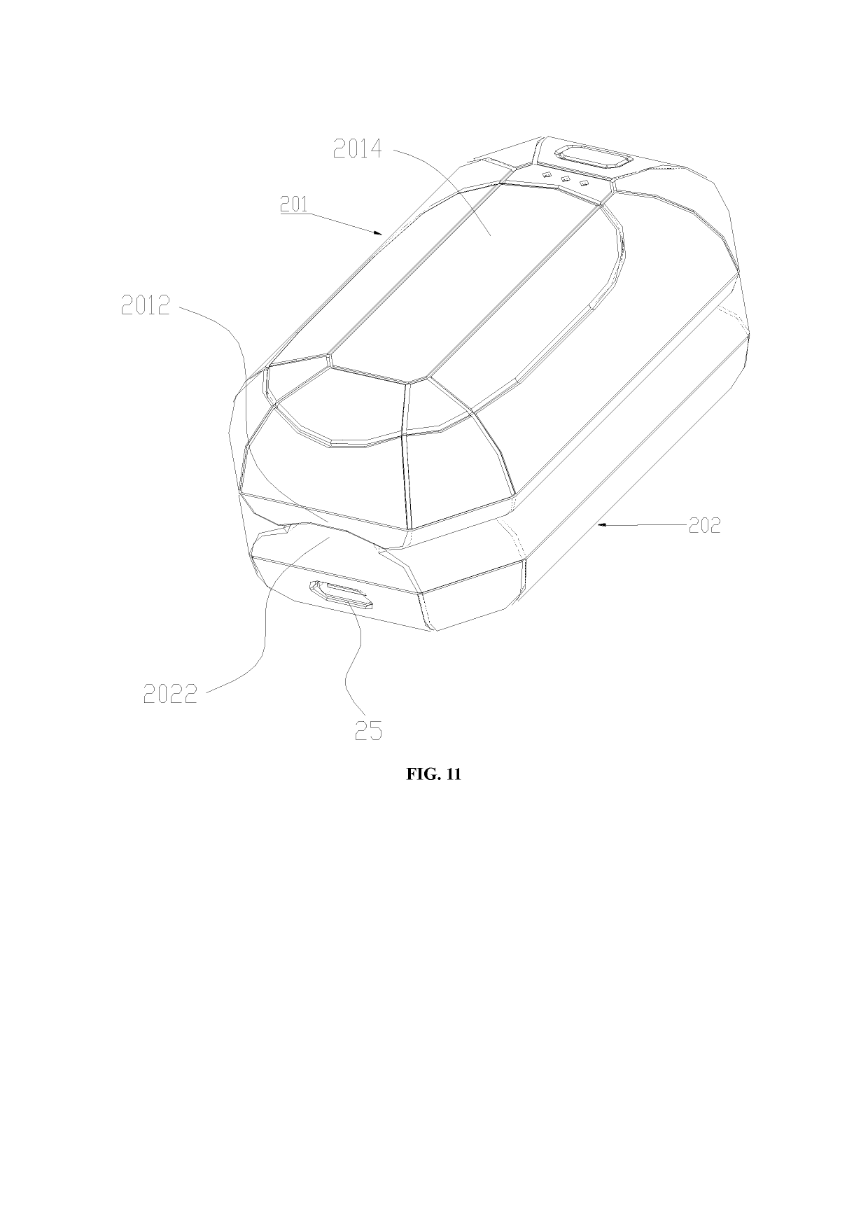

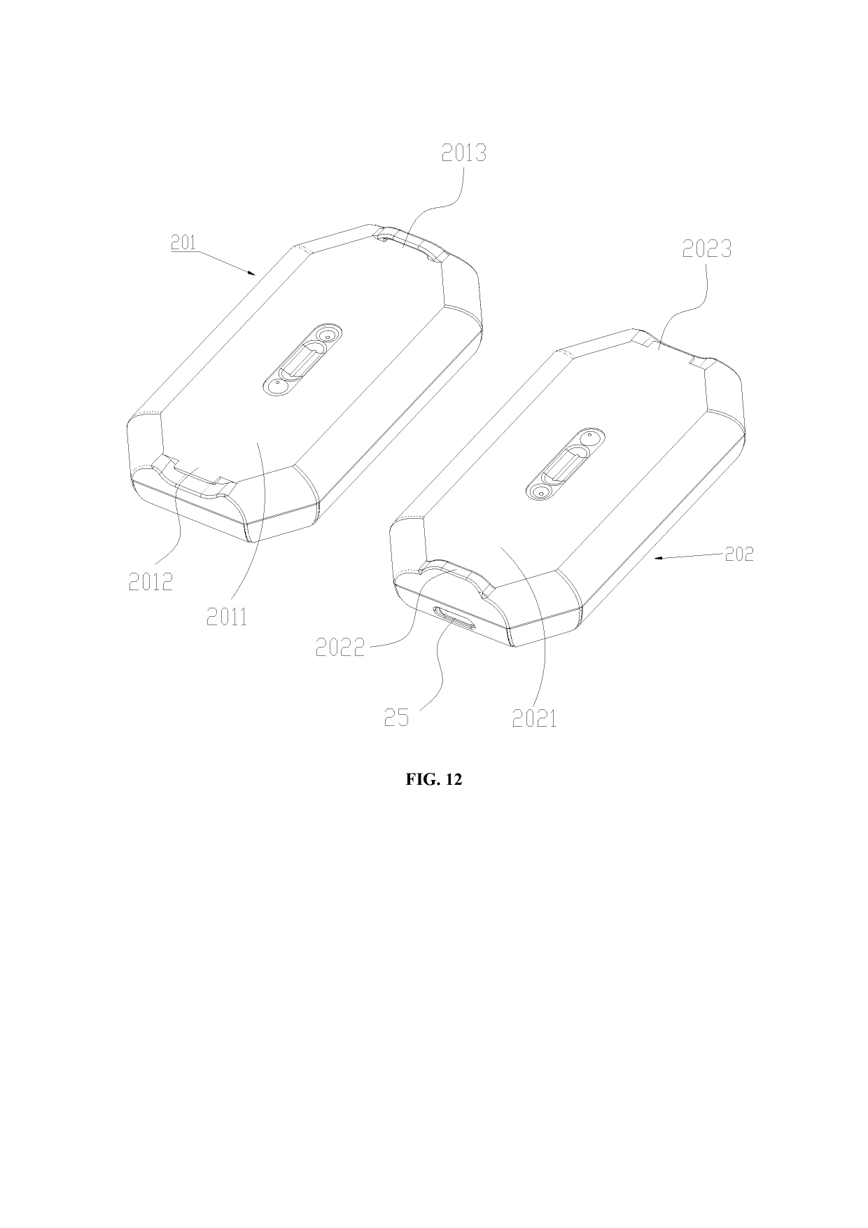

Referring to FIG. 11 to FIG. 12, in an embodiment, there are two hand warmers 2: a first hand warmer 201 and a second hand warmer 202. Correspondingly, there are two receiving chambers 3.

In an embodiment, the first hand warmer 201 is detachably connected to the second hand warmer 202, so that the first hand warmer 201 and the second hand warmer 202 are combined into a whole. When a user needs to warm both hands/multiple body parts at the same time, the first hand warmer 201 can be separated from the second hand warmer 202, so that the first hand warmer 201 and the second hand warmer 202 can be used for warming both hands/multiple different body parts separately. When a user needs to warm one hand/one body part, the first hand warmer 201 and the second hand warmer 202 can be combined into a whole to enlarge a warming area of the hand warmer for one hand/one body part, thus enhancing the warming effect. Moreover, when a user needs to carry or store the hand warmer, the user can selectively carry and store the first hand warmer 201 and the second hand warmer 202 separately according to an actual situation, or selectively combine the first hand warmer 201 with the second hand warmer 202 into a whole for carrying and storage, to meet the needs of the user in multiple scenarios, which greatly improves the user experience.

Specifically, the first hand warmer 201 includes a first outer surface 2014 and a first connecting surface 2011 opposite to the first outer surface 2014; a first connecting portion is arranged on the first connecting surface 2011; the second hand warmer 202 includes a second outer surface and a second connecting surface 2021 opposite to the second outer surface; and a second connecting portion is arranged on the second connecting surface 2021; the first connecting portion is connected to the second connecting portion in one of the following manners: a buckle, a hook and loop fastener, and magnetic suction of a plurality of magnetic suction assemblies, so that the first connecting inner surface is connected to the second connecting inner surface; and the first hand warmer 201 and the second hand warmer 202 are combined into a whole.

In an embodiment, the first connecting portion includes a first clamping slot 2013 and a first buckle 2012; the first clamping slot 2013 and the first buckle 2012 are respectively located at two ends of the hand warmer 2; the second connecting portion includes a second clamping slot 2022 and a second buckle 2023; the first buckle 2012 is clamped with the second clamping slot 2022 when the first hand warmer 201 and second hand warmer 202 are assembled together; and the second buckle 2023 is clamped with the first clamping slot 2013 when the first hand warmer 201 and second hand warmer 202 are assembled together. In this way, the buckle structures at the two ends can assemble the first hand warmer 201 with the second hand warmer 202 stably together. Of course, in other embodiments, the first connecting portion may include two clamping slots, and the second connecting portion may include two buckles adapted to the clamping slots. Alternatively, both the first connecting portion and the second connecting portion can be hook and loop fasteners, magnets, or the like, and the first hand warmer 201 and the second hand warmer 202 can be detachably connected to each other.

In an embodiment, the hand warmer 2 is further provided with a second charging interface 25 for an external power cable. The second charging interface 25 is used for being externally connected to a power supply. After the power supply is connected, the second battery 21 can be charged through the second circuit board 23. In order to increase the charging speed when the charging case 1 has low battery, both the charging case 1 and the hand warmer 2 can be charged separately through a power cable. When the hand warmer 2 is charged using the second charging interface 25, the power of the charging case 1 will not be consumed, so that when a user goes out, the charging case 1 can keep sufficient power for use. Specifically, both the first charging interface 136 and the second charging interface 25 can be Type-C interfaces, Micro USB interfaces, Lightning interfaces, direct current interfaces, and the like.

In an embodiment, the capacity of the first battery is greater than the capacity of the second battery, such as the capacity of the first battery 18 is at least twice that of the second battery 21, so that the charging case 1 can simultaneously fully charge at least two hand warmers 2. Of course, in other embodiments, the capacity of the first battery 18 may be at least three times, four times, etc. greater than the capacity of the second battery 21, so that the charging case 1 can simultaneously charge two hand warmers 2 at least twice, making it convenient for a user to use the charging case when the user goes out.

In some embodiments, the case body 10 of the portable hand warmer charging case includes an opening 101, the hand warmer 2 is put into or inserted into the receiving chamber 10 from the opening 101. In present embodiment, the opening 101 is unsealed, i.e., it is open without a cover, such case body 10 with unsealed opening 101 is more convenient for use, and after use, it can be stored to maintain clean.

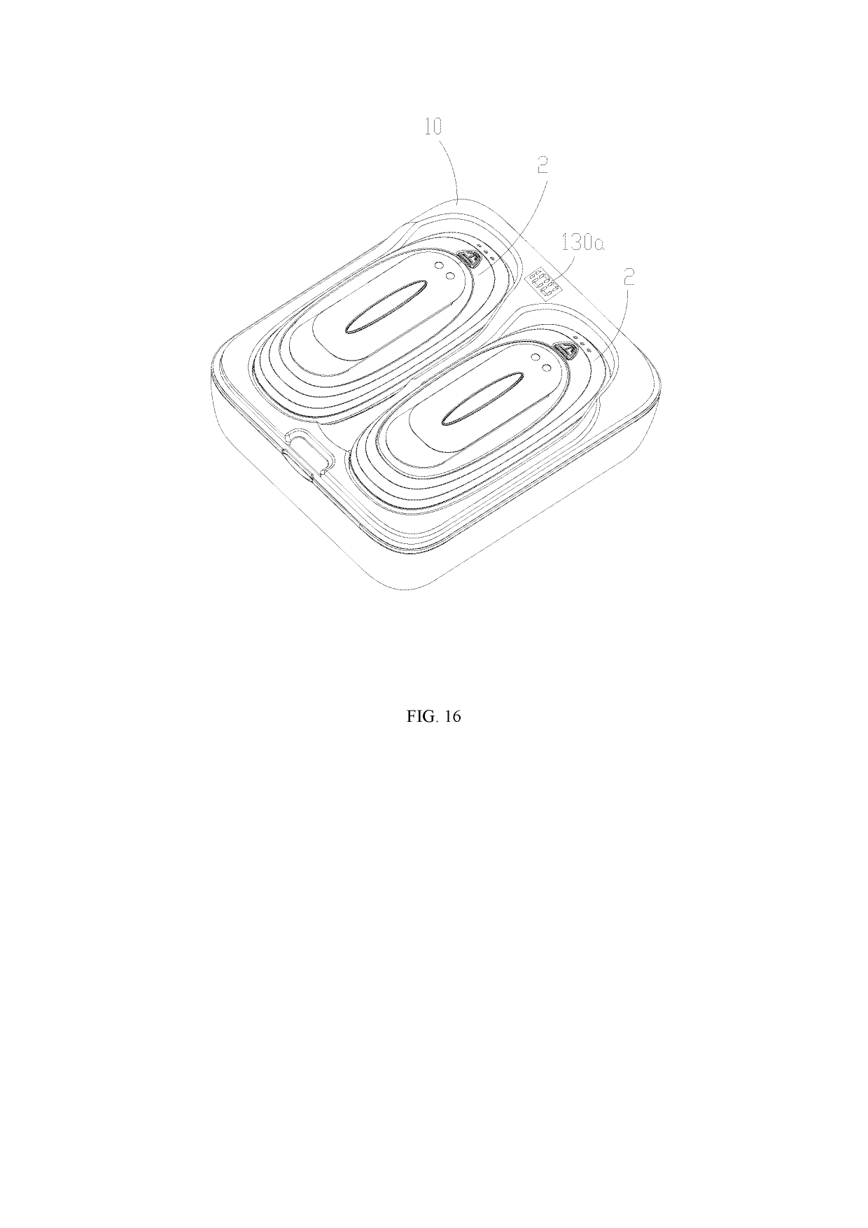

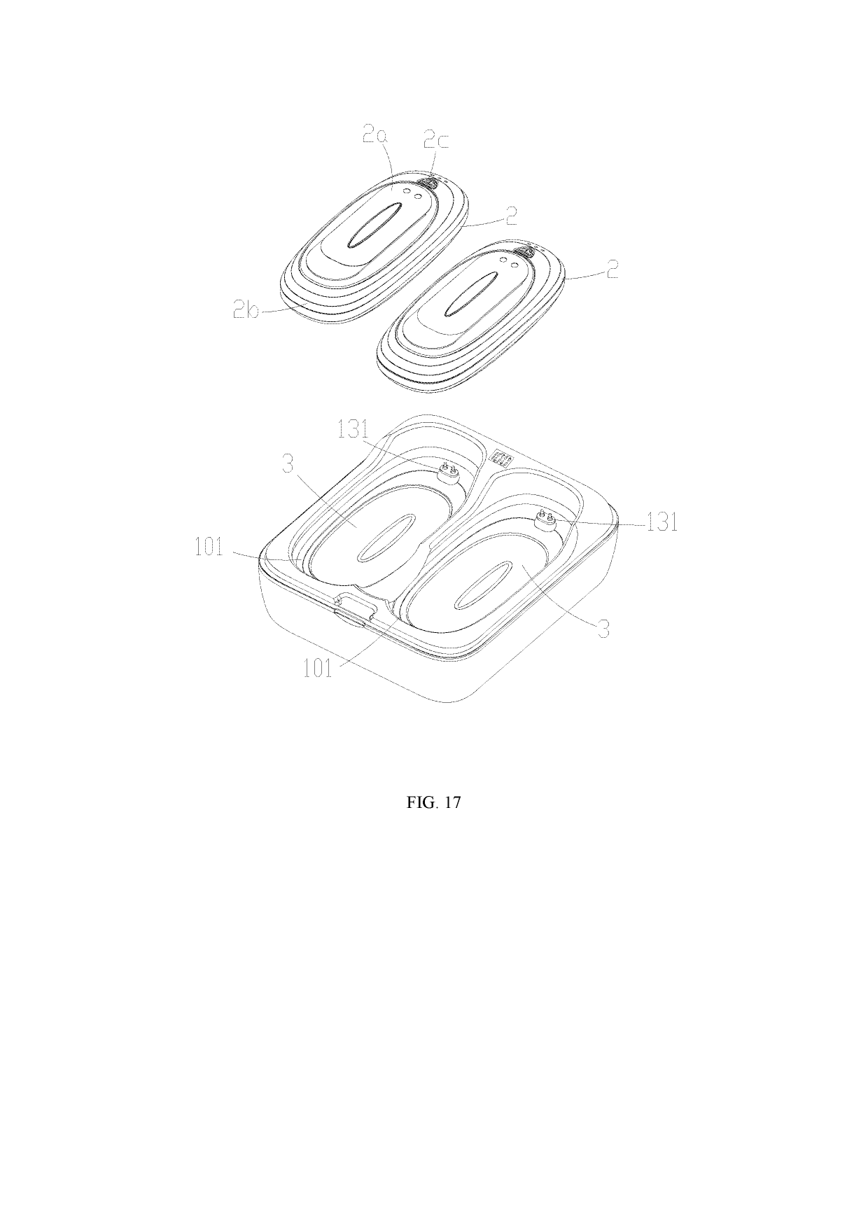

In particular, referring to FIGS. 16-18, in this embodiment, the opening 101 faces upwards, the conductive probe 131 faces a direction of the opening 101 to adapt for an inserting direction of the hand warmer 2, the hand warmer 2 has the second charging unit 24 to adapt for the conductive probe 131. The case body 10 is provided with an indicator unit 130a configured to indicate a battery level state of the first battery 18 and/or a charging state of the hand warmer 2, the indicator unit 130a is displayed facing an upper surface of the case body 10. The receiving chamber 3 is configured for the hand warmer 2 being placed flat in the receiving chamber 3, after the hand warmer 2 is placed in the receiving chamber 3, an upper surface 2a of the hand warmer 2 faces upwards, and is exposed at the opening 101.

The indicator unit 130a is electrically connected to the first circuit board 13, and the indicator unit 130a is a display screen, and display number representing the charging state, for example, when 100 is displayed, it represents the charging state is finished 100%. It can be understood that the display screen 130a may include a light-emitting light board 130b electrically connected to the first circuit board 13 and a display cover 130c arranged on the light-emitting light board 130b. The light-emitting light board 100b has multiple light-emitting elements 130d, and the first circuit board 13 controls the digital display on the display screen 130a by controlling the light emission or turn off of the multiple light-emitting elements 130d. The upper surface 2a of the hand warmer 2 can have a display 2c, such display 2c can display the state of the hand warmer 2, such as battery level state or abnormal alarm information; additionally, switch element for controlling the hand warmer 2 can be set on the hand warmer 2.

In the above embodiment, the number of the receiving chamber 3 is two, the number of the openings 101 are two, and the two receiving chambers 3 are parallel with each other, the two openings 101 are parallel with each other and are in communication with each other, such that two hand warmers 2 can be in charging state at a same time.

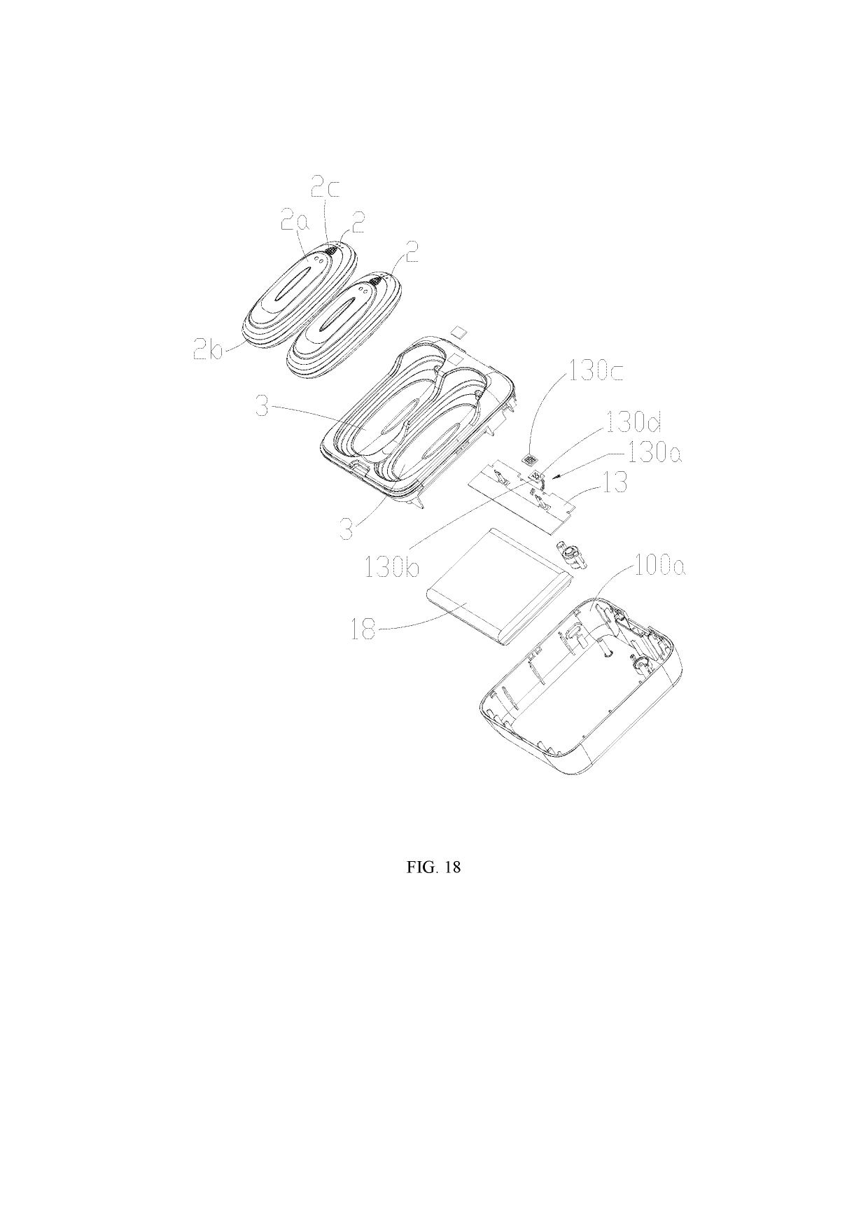

In FIG. 18, it shows the charging case substantially includes a housing 100a, the first circuit board 13, the first battery 18 and the receiving chambers 3, the receiving chambers 3 are integrally formed into one piece, and the one piece served as the upper shell of the case body 10, the first circuit board 13 and the first battery 18 are mounted in the housing 100a, these can be the same as other embodiments.

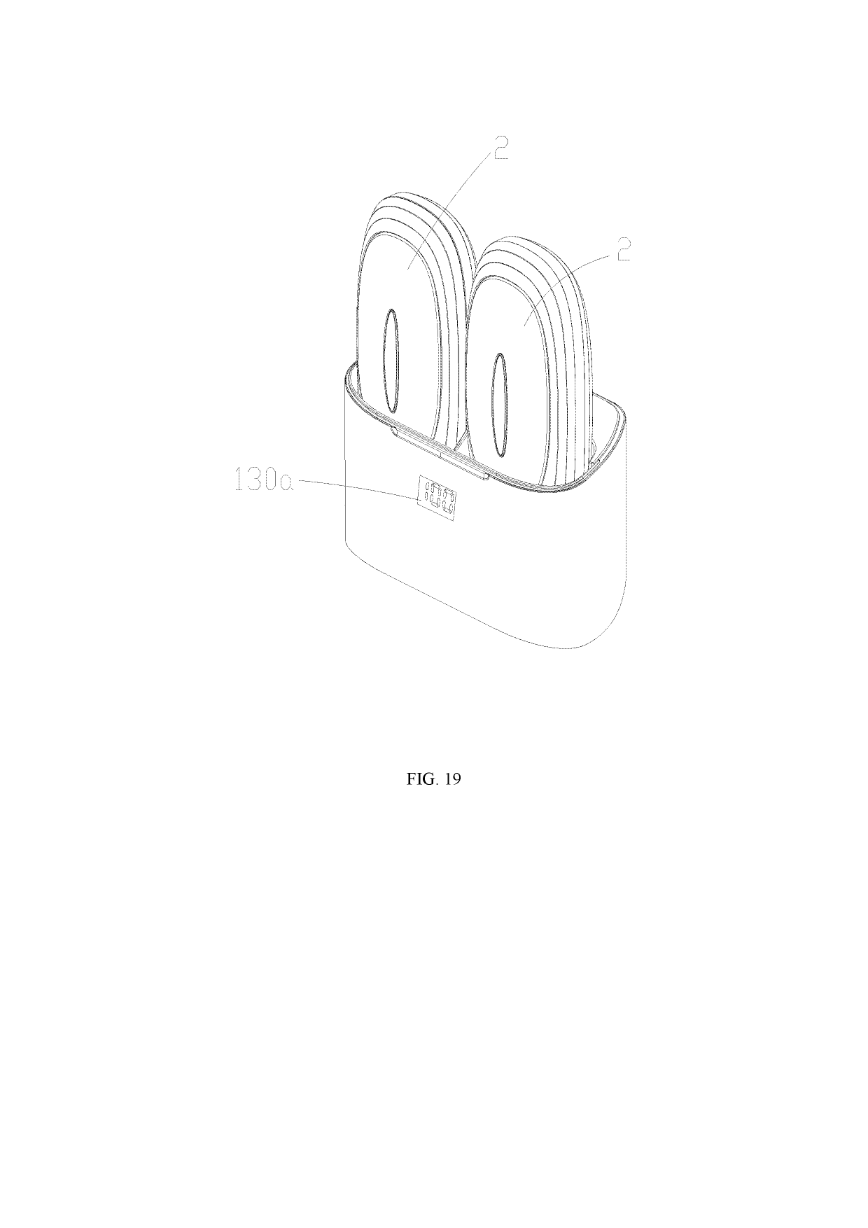

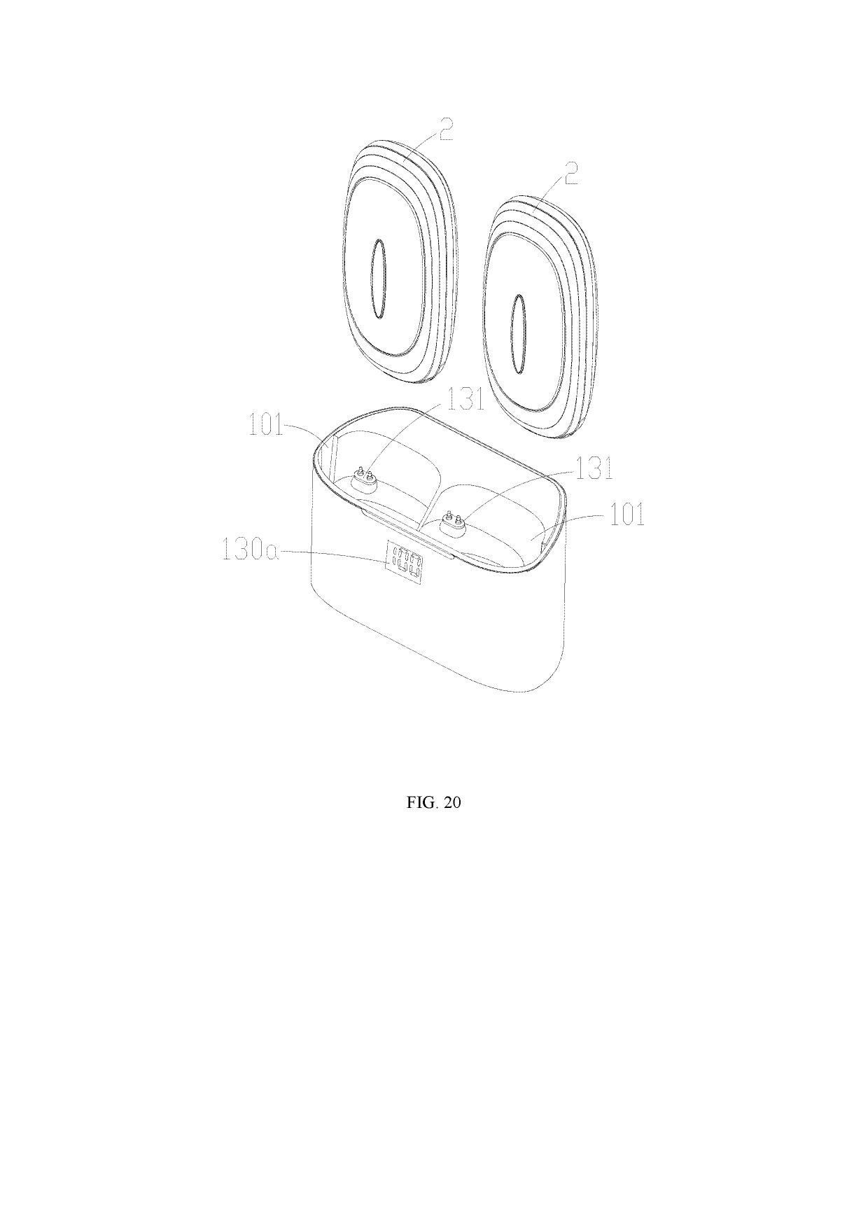

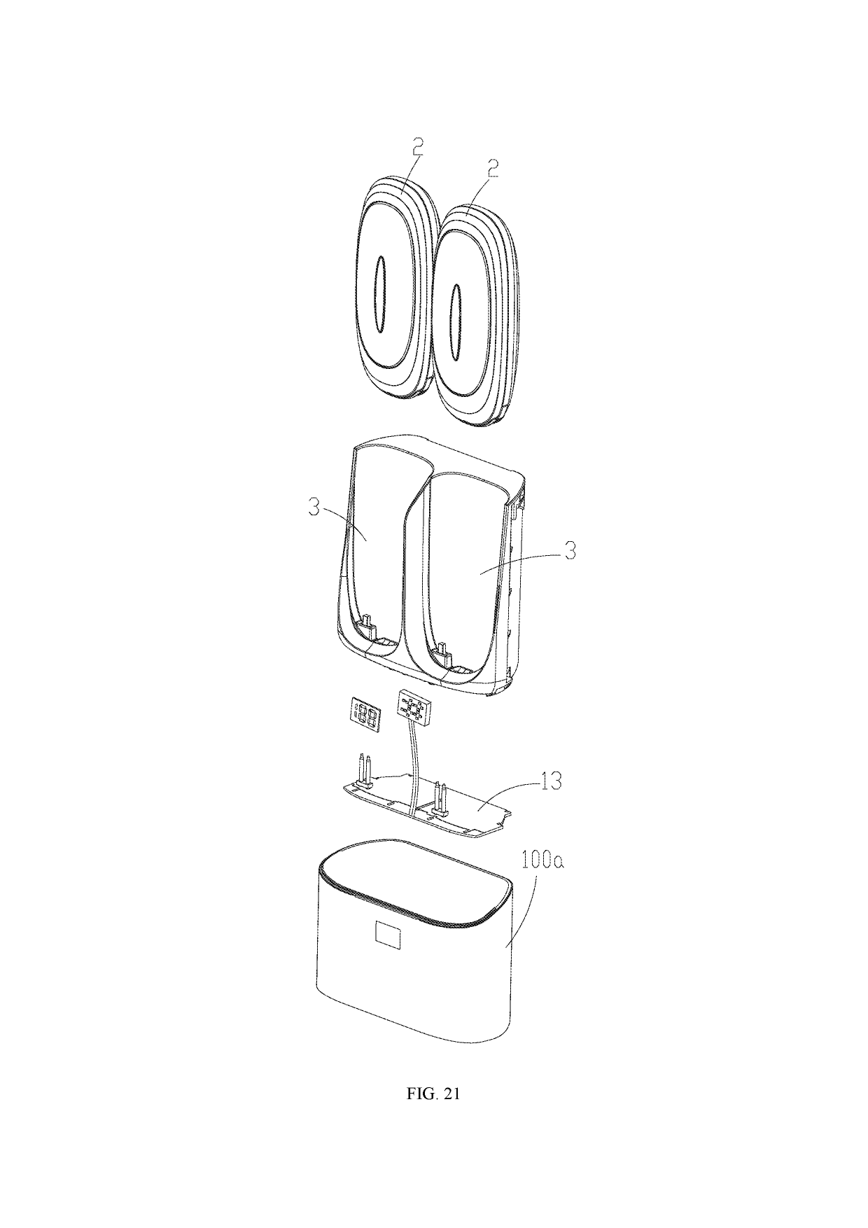

Referring to FIGS. 19-21, in this embodiment, the opening 101 faces upwards, the case body 10 is provided with an indicator unit 130a configured to indicate a battery level state of the first battery 18 and/or a charging state of the hand warmer 2, the indicator unit 130a is displayed facing a side surface of the case body 10. The hand warmer 2 includes an upper surface 2a and an end surface 2b perpendicular to the upper surface 2a, and the receiving chamber 3 is configured for the hand warmer 2 being vertically inserted into the receiving chamber 3, after the hand warmer 2 is placed in the receiving chamber 3, the end surface of the hand warmer 2 faces upwards, and a part of the upper surface 2a is exposed at the opening 101.

In the above embodiment, the number of the receiving chamber 3 is two, the number of the openings 101 are two, and the two receiving chambers 3 are parallel with each other, the two openings 101 are parallel with each other and are in communication with each other, such that two hand warmers 2 can be in charging state at a same time.

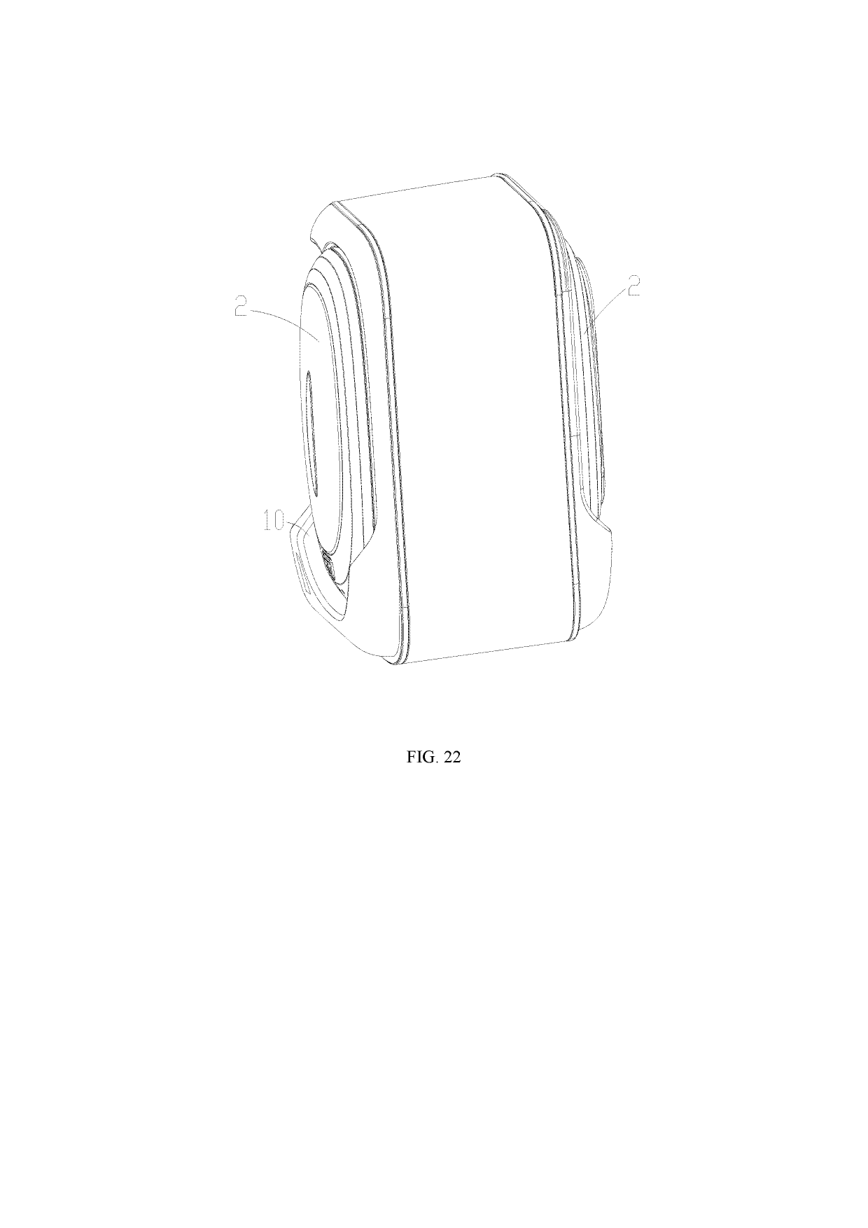

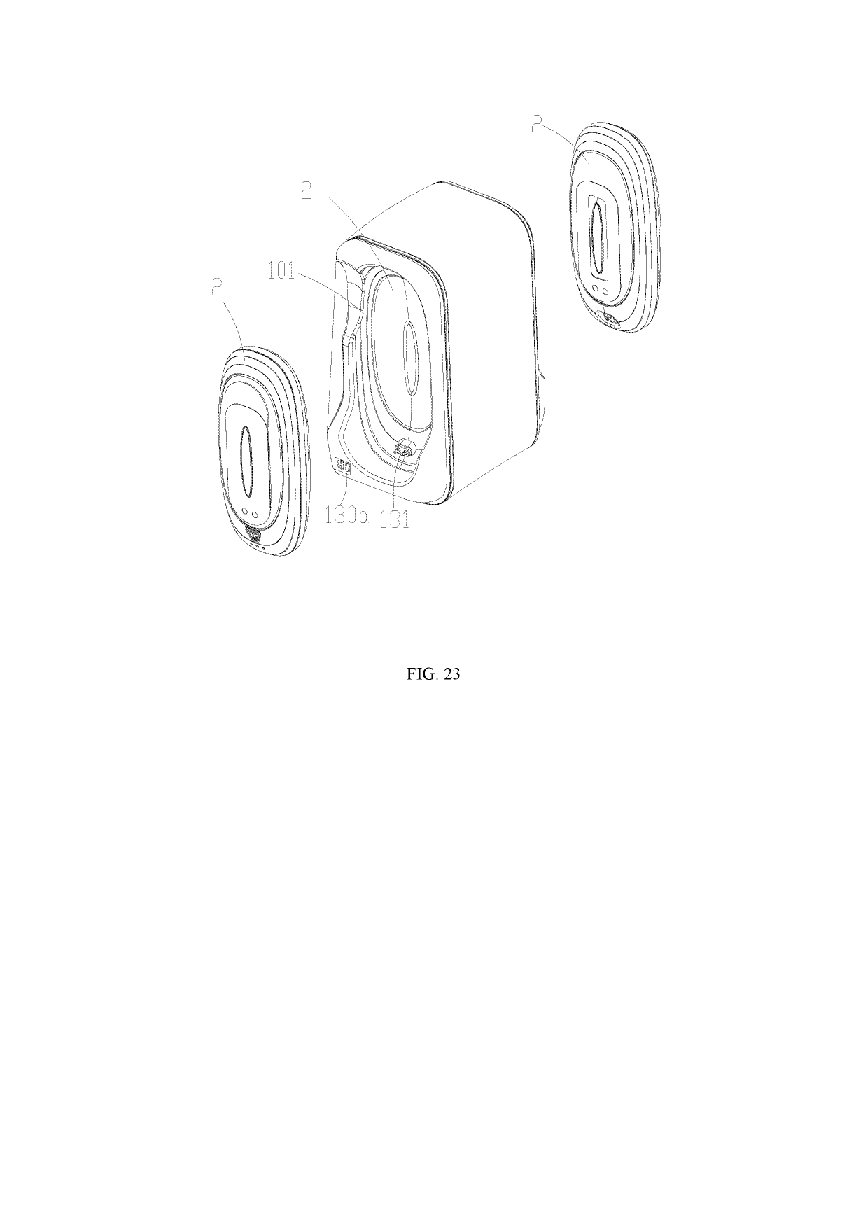

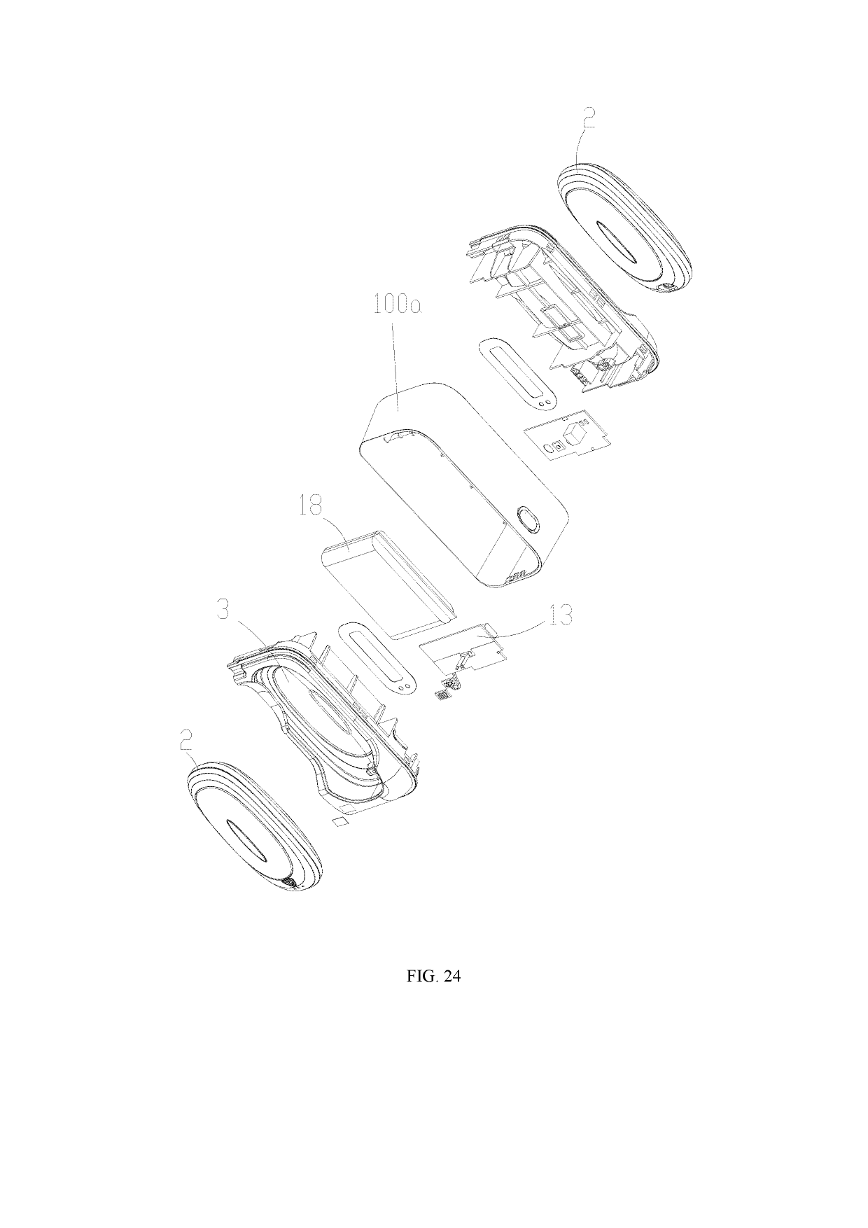

Referring to FIGS. 22-24, in this embodiment, the opening 101 faces a side surface of the case body 10, the case body 10 is provided with an indicator unit 130a configured to indicate a battery level state of the first battery 18 and/or a charging state of the hand warmer 2, the indicator unit 130a is displayed facing the side surface of the case body 10. In this embodiment, the receiving chamber 3 is configured for the hand warmer 2 being placed to the receiving chamber 3 from one side of the case body 10, and after the hand warmer 2 is placed in the receiving chamber 3, an upper surface of the hand warmer 2 faces the side surface of the case body 10, and is exposed at the opening 101.

In the above embodiment, the number of the receiving chamber 3 is two, the number of the openings 101 are two, and the two receiving chambers 3 are opposite to each other, the two openings 101 are opposite to each other, two hand warmers 2 can be inserted from opposite side at a same time.

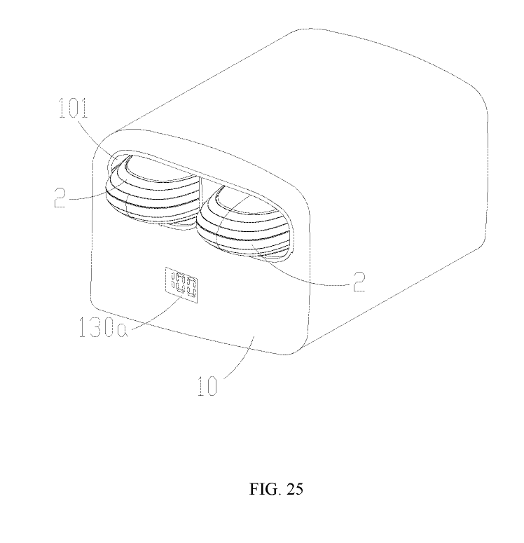

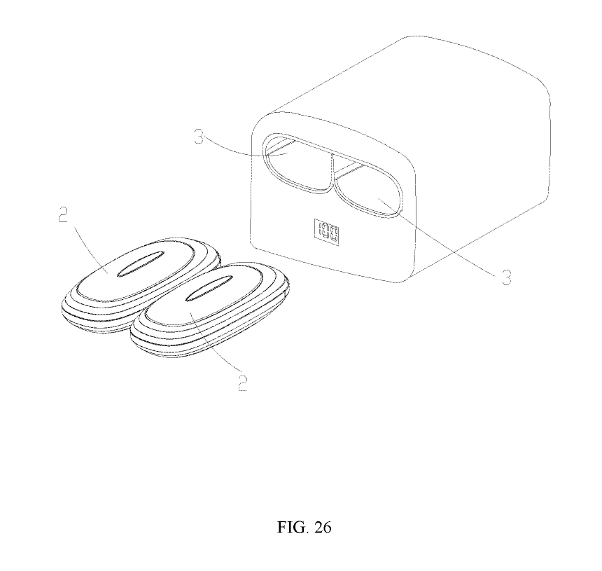

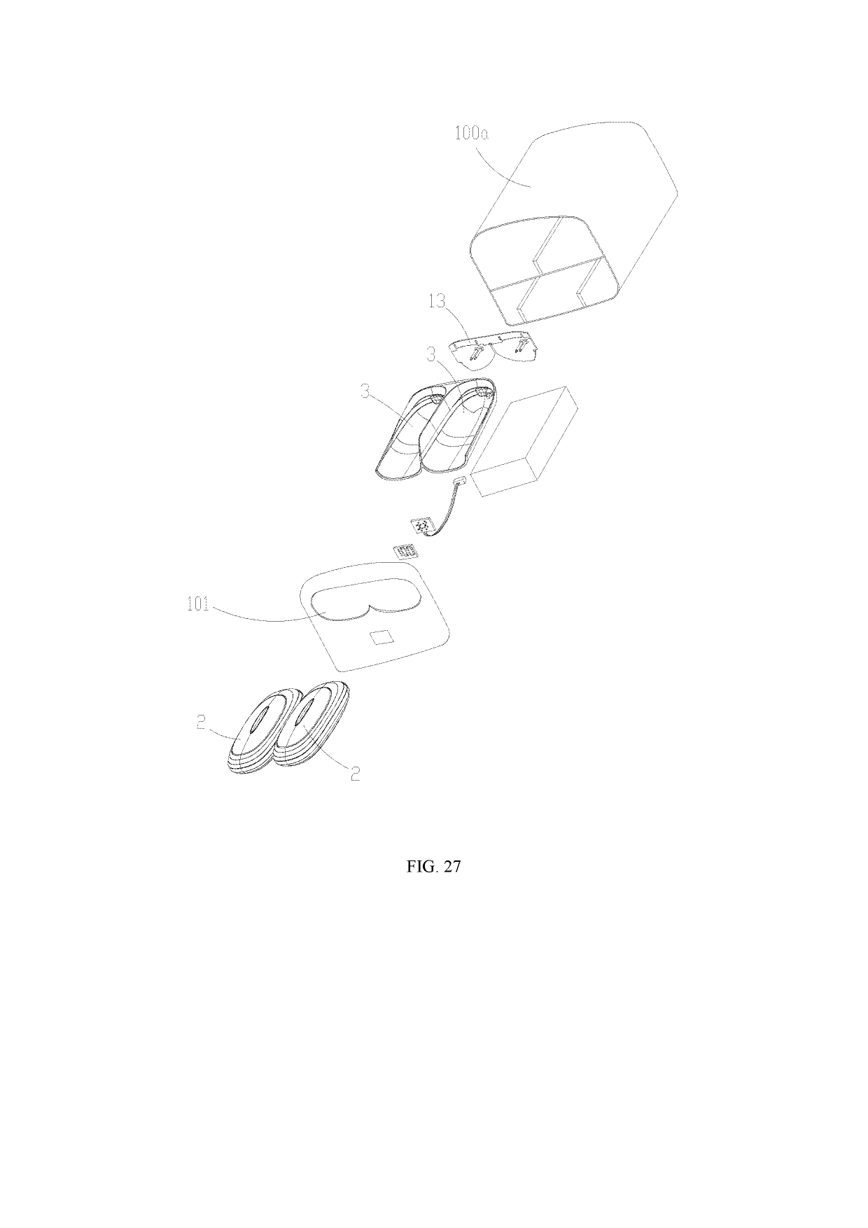

Referring to FIGS. 25-27, in this embodiment, the receiving chamber 3 is a groove with a certain depth, which only has an opening 101 at one end to expose the outside, the opening 101 faces a side surface of the case body, the case body 10 is provided with an indicator unit 130a configured to indicate a battery level state of the first battery 18 and/or a charging state of the hand warmer 2, the indicator unit 130a is displayed facing the side surface of the case body 10. The hand warmer 2 includes an upper surface 2a and an end surface 2b perpendicular to the upper surface 2a, after the hand warmer 2 is placed in the receiving chamber 3, the end surface 2b of hand warmer 2 is exposed at the opening 101, and the upper surface 2a is not exposed at the opening 101.

In the above embodiment, the number of the receiving chamber 3 is two, the number of the openings 101 are two, and the two receiving chambers 3 are parallel with each other, the two openings 101 are parallel with each other and in communication with each other, such that two hand warmers 2 can be in charging state at a same time.

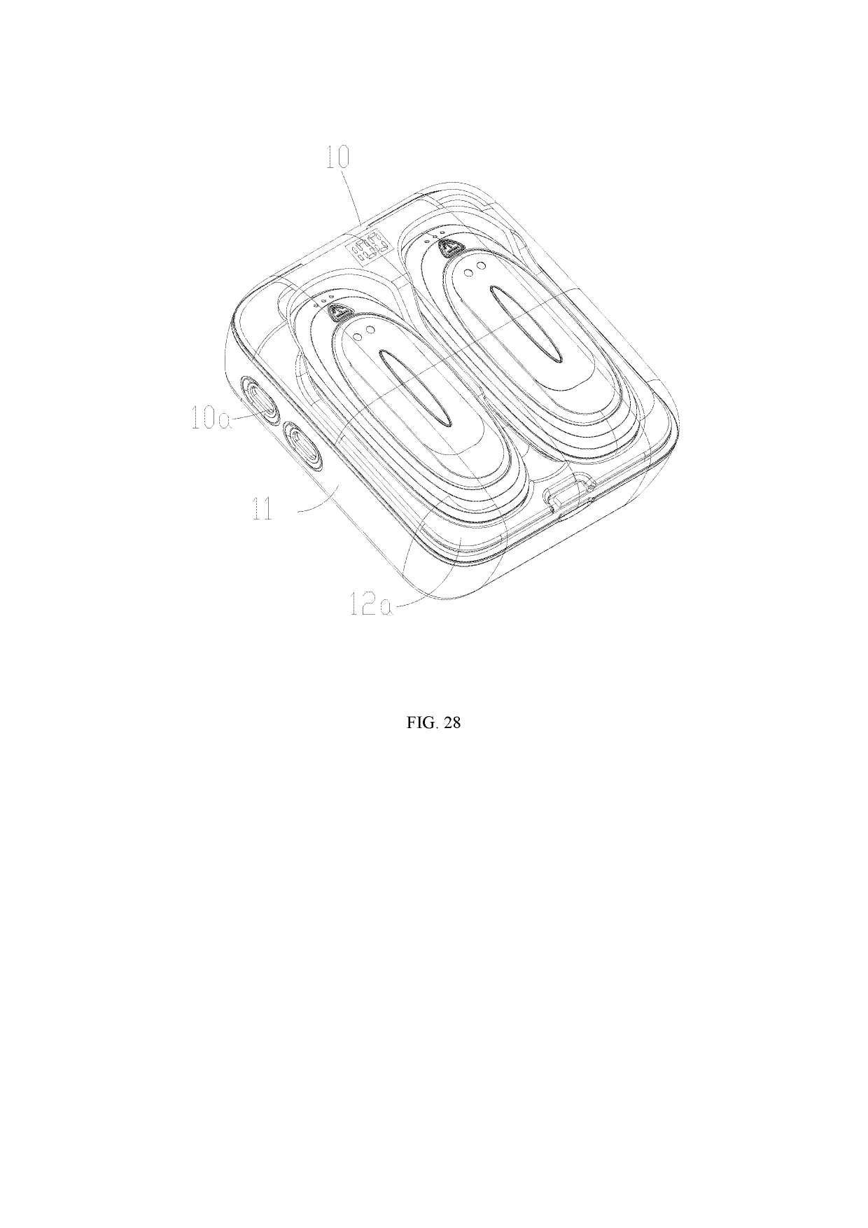

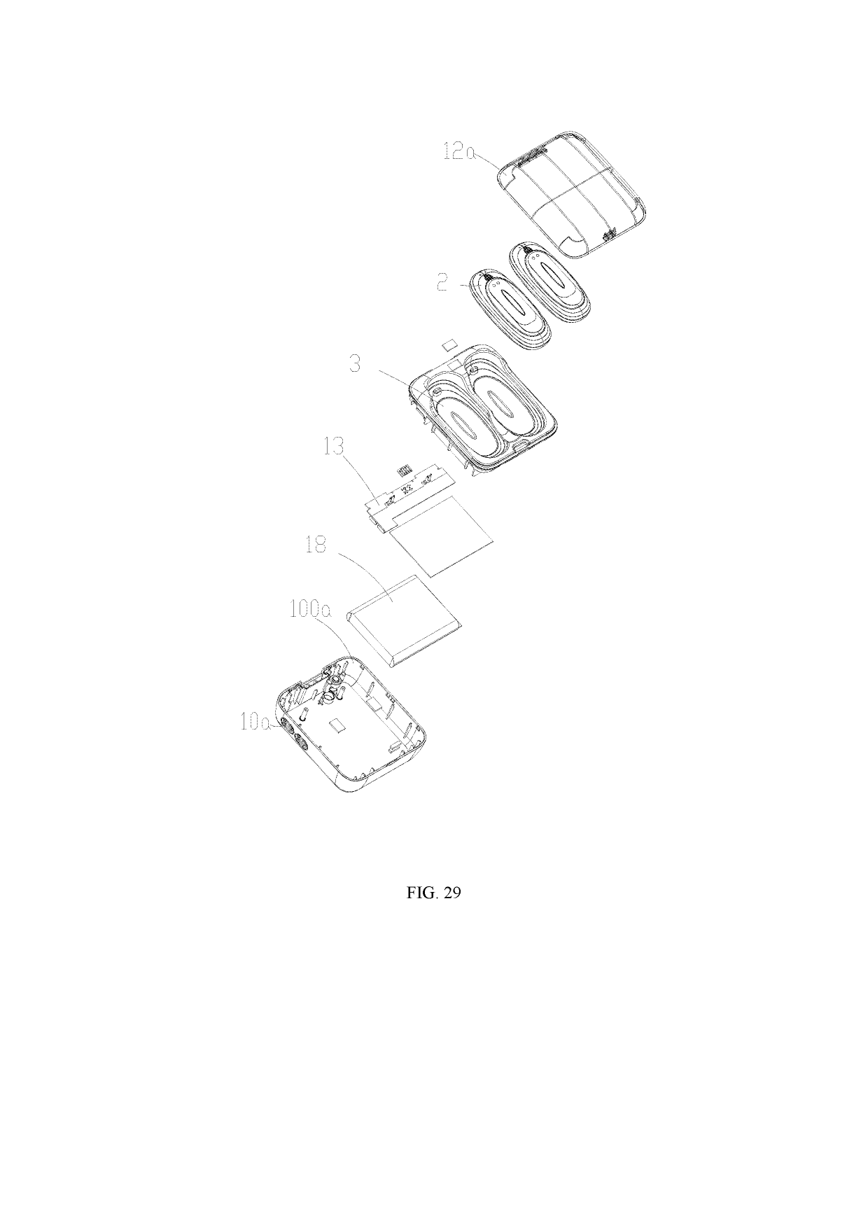

Referring to FIGS. 28-29, in another embodiment, the case body 10 of the portable hand warmer charging case includes a main body 11 and a cover body 12a connected to the main body 11 to form the receiving chamber 3. The main body 11 includes the above mentioned housing 100a, the first circuit board 13, the first battery 18 and the receiving chamber 3. The cover body 12a is transparent, so that the relevant functions, battery status, or abnormal alarm information of the hand warmer 2 can be observed through the transparent cover body 12a.

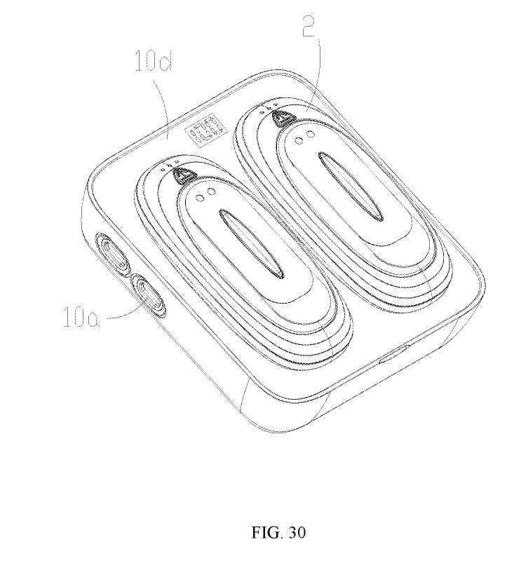

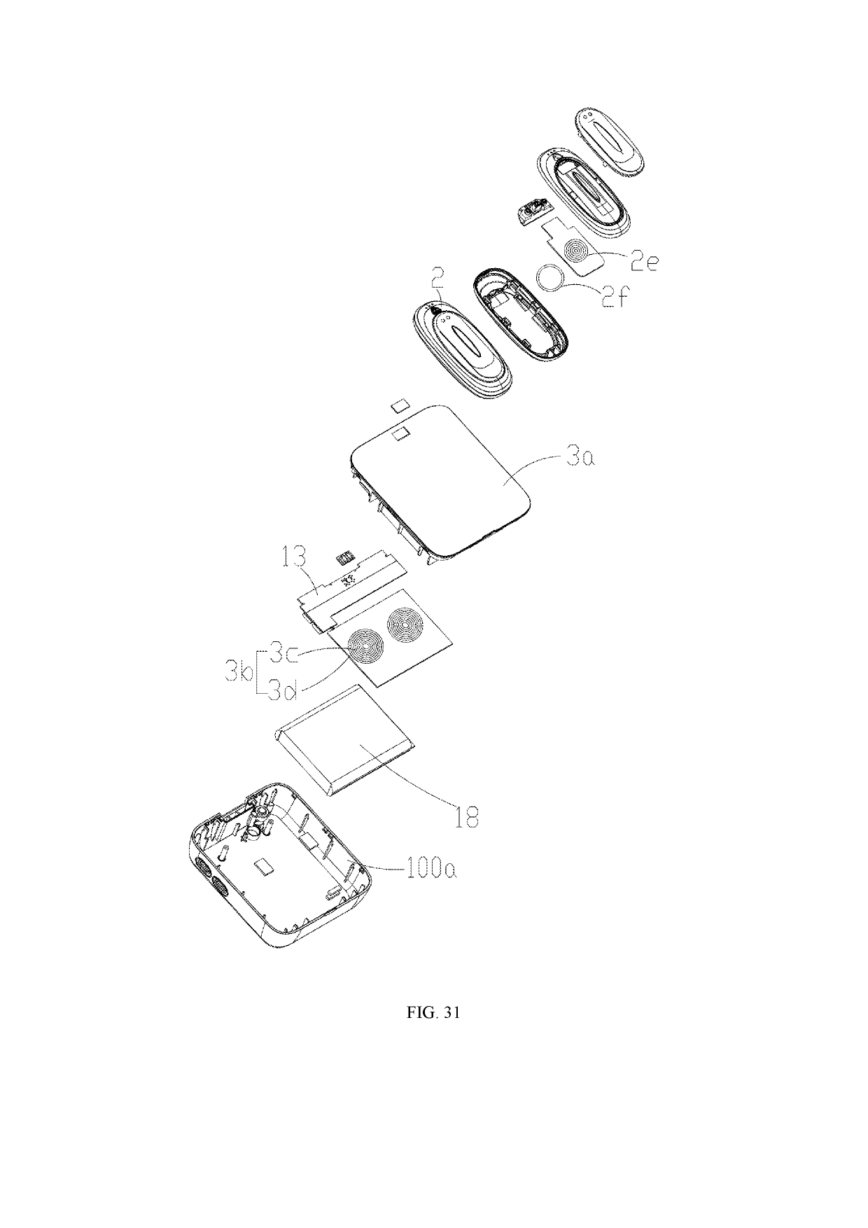

Referring to FIGS. 30-31, in further another embodiment, it is a portable hand warmer charging device 10d without a cover, but also has the housing 100a, the first circuit board 13, the first battery 18, and the portable hand warmer charging device 10d has a receiving portion 3a, the receiving portion 3a is only a flat portion, not a chamber illustrated in above embodiments, when the hand warmer 2 is placed thereon, it can be charged through contact or through wireless manner, in this embodiment, wireless charging is used as a schematic illustration.

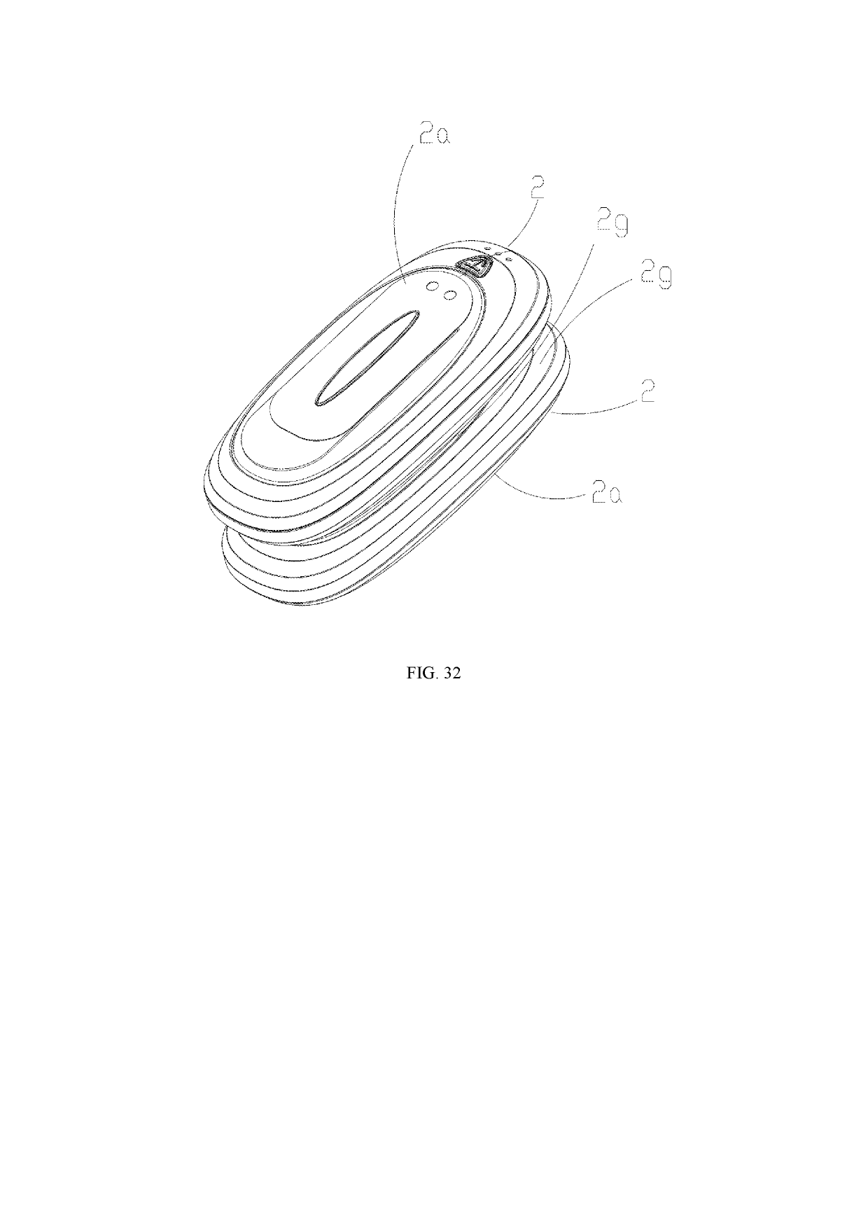

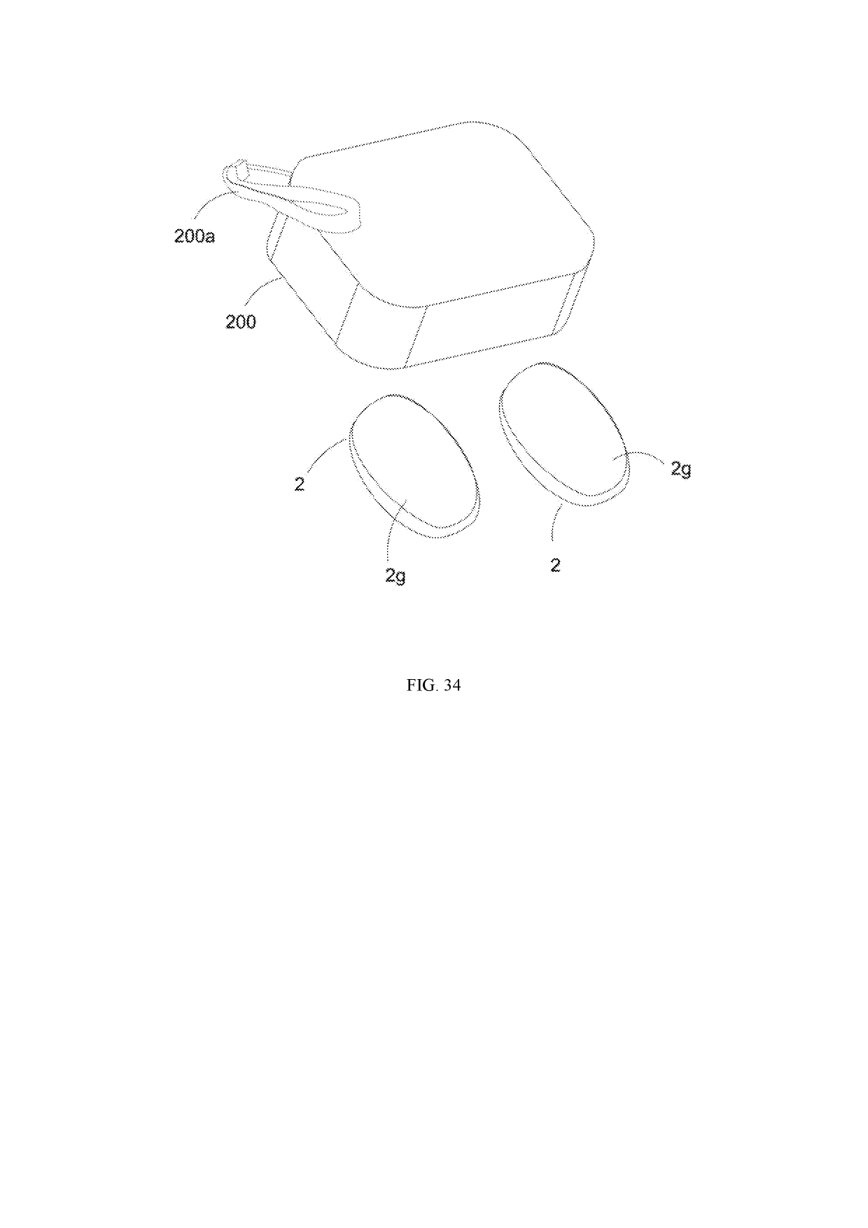

Referring to FIGS. 31 and 32, a wireless charging assembly 3b having a wireless charging coil 3c is arranged under the placement portion 3a, and is electrically connected to the first circuit board 13. The wireless charging assembly 3b further includes a first magnetic member 3d, the first magnetic member 3d can be circular magnetic member, in detail, the first magnetic member 3d surrounds the wireless charging coil 3c. The hand warmer 2 has a second magnetic member 2f and a wireless charging coil 2e arranged therein, the second magnetic member 2f corresponds to the first magnetic member 3d. The hand warmer 2 can be attached to the placement portion 3a by abstraction of the first magnetic member 3d and the second magnetic member 2f, and after the hand warmer 2 is abstracted to the placement portion 3a, the wireless charging coil 3c can correspond to the wireless charging coil 2e to charge the hand warmer 2. Furthermore, in present embodiment, each of the two hand warmers 2 has the second magnetic member 2f, the two warmers 2 can be combined as a whole by attraction between the second magnetic members 2a, in this way, the second magnetic member 2f is not only used to attach the hand warmer 2 to the placement portion 3a of the portable hand warmer charging device 10d when in charging, but also used to connect the two warmers 2 to be a combination. In this way, a design cost can be reduced, and a user experience can be better. In present embodiment, the two hand warmers 2 are combined up and down by lower surfaces 2g attracted together, and upper surface 2a can face outside, the upper surface 2a can warm hand, the combination is more convenient to carry, and can increase the hand warming temperature. Please refer to FIGS. 32 and 34, two hand warmers 2 can be disassembled and connected, each hand warmer 2 is equipped with a heating element, and each hand warmer 2 has an upper surface 2a that heats up externally and a lower surface 2g opposite to the upper surface 2a, during hand warmer operation, each hand warmer 2 heats up for warming separately/individually through the upper surface 2a, or the two hand warmers 2 are stacked by pressing the lower surfaces together to form a combined unit for warming.

Referring again to FIG. 28 or 30, the portable hand warmer charging case or the portable hand warmer charging device 10d further includes one or more charging interfere 10a which is electrically connected to the first circuit board 13, and is configured for charging other devices which are not the hand warmers 2. The charging interface 10a can be a type-C female socket interface, a USB type-A female socket interface, etc. This embodiment mainly illustrates the charging interface 10a as a type-C female socket interface. By setting the charging interface 10a, the hand warmer charging device 10d can also charge the hand warmer 2 through an external data cable, or charge other electronic devices such as mobile phones through an external data cable, making it versatile and providing a better user experience.

Based on the structure of the portable hand warmer charging case 1 in the above embodiments, in some broader embodiments, the physical structure of the charging case 1 of the present invention, after peeling off its charging electrical components, can essentially serve as a portable hand warmer storage box for centralized storage and carrying of multiple hand warmers.

Specifically, referring to FIGS. 32-34, the present application also provides a hand warmer assembly, which includes at least two hand warmers 2 and a storage box 200. Among them, each of the hand warmers 2 is equipped with a heating element 22, and each hand warmer 2 has an upper surface 2a that heats up to the outside and a lower surface 2g opposite to the upper surface 2a. During hand warmer operation, each hand warmer 2 heats up to the outside, such as to warm hands separately through the upper surface 2a, or the at least two hand warmers 2 are stacked in an up and down manner by stacking the lower surfaces 2g together to form a combined unit, and the combined unit can heat up to outside through their upper surfaces 2a, with one upper surface 2a at the top, the other upper surface 2a at the bottom, i.e, the combined unit forms double warming surfaces at the up and down manner relative to a single hand warmer 2.

The storage box 200 includes a case body 10, which is provided with at least one receiving chamber 3 for placing at least two hand warmers 2. The case body 10 is provided with an opening 101 corresponding to the receiving chamber 3, and at least two hand warmers 2 are respectively placed into at least one of the receiving chambers 3 through the opening 101. By configuring at least two detachable hand warmers 2, and placing both hand warmers 2 in the receiving chamber 3 of the storage box 200, at least two hand warmers 2 can be uniformly and orderly placed into the receiving chamber 3 for centralized storage through the opening 101 when idle or carried out.

Their lower surfaces 2g can be adhered to each other (such as by using their respective first and second connection parts for snap fit, Velcro, or magnetic connection), forming a combined unit of upper and lower stacking. This can allow the upper surfaces 2a of the two hand warmers 2 to heat up together for warming a hand when the hand holds the combined unit, further expanding the heating area and improving the overall heating effect, so that the two can be combined into a whole for users to warm up; the setting of two or more detachable hand warmers 2 breaks the limitations of traditional single body hand warmers, easily meeting the needs of users to heat their hands separately or share heating with others, significantly improving the flexibility of use and the user's heating experience. The storage box effectively solves the technical pain points of difficult unified management and easy scattering or loss when carrying multiple hand warmers, greatly improving the overall portability and storage cleanliness of the product.

On top of the basic structure of the storage box 200 mentioned above, referring to FIGS. 1-10, when it is given a charging function (i.e. as the portable hand warmer charging compartment 1 mentioned above), the case body 10 is equipped with a first circuit board 13, a first charging unit 131, and a first battery 18, both of which are electrically connected to the first circuit board 13. Correspondingly, at least two of the hand warmers 2 are equipped with a second circuit board 23, a second battery 21, and a second charging unit 24. The heating element 22, the second battery 21, and the second charging unit 24 are all electrically connected to the second circuit board 23. When the hand warmer 2 is placed in the receiving chamber 3, the second charging unit 24 is close to or in contact with the first charging unit 131. The first charging unit 131 is used to output the electrical energy output from the first circuit board 13 to the second charging unit 24. The second charging unit 24 charges the second battery 21 with the electrical energy output from the first charging unit 131 through the second circuit board 23. Among them, the first charging unit 131 can be a conductive probe set at the bottom of the receiving chamber 3, and the second charging unit 24 is a charging contact located at the bottom of the hand warmer 2.

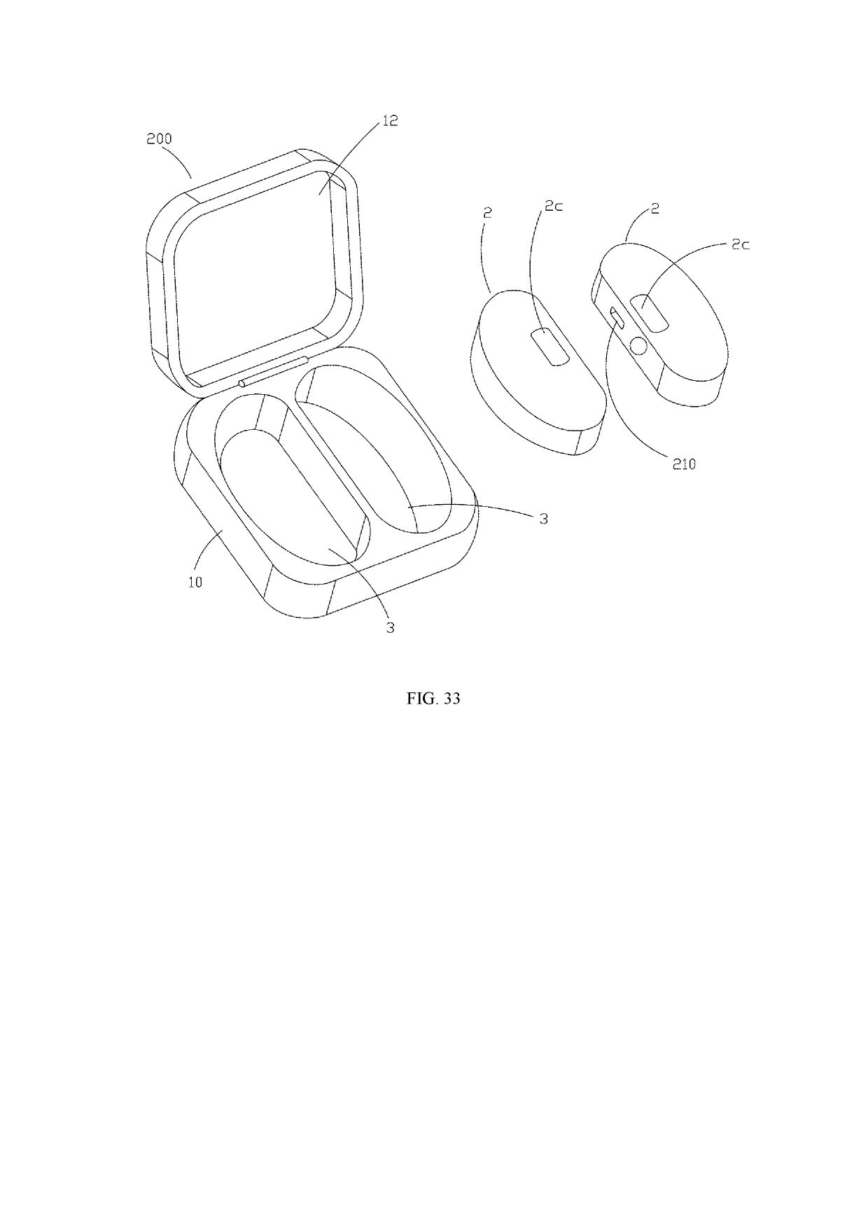

In order to achieve independent and orderly storage of multiple hand warmers 2, in some embodiments, the case body 10 is provided with a receiving chamber 3 for placing at least two hand warmers 2. When multiple receiving chambers 3 are set, they are separated and arranged (see FIG. 33). For example, in this embodiment, there are two receiving chambers 3, which are arranged in parallel, and the corresponding two openings 101 are also arranged in parallel. Correspondingly, there are two hand warmers 2, namely the first hand warmer 201 and the second hand warmer 202; when storing or charging, the first hand warmer 201 and the second hand warmer 202 are separated and placed in the corresponding separate placement cavities 3. When taken out for use, the first hand warmer 201 and the second hand warmer 202 can be detachably connected (as mentioned above, the first connection part and the second connection part can be fastened, Velcro or magnetically connected), so that the two can be combined into a whole for users to keep warm.

Furthermore, in order to enhance the user experience of the storage box, the case body 10 includes a main body 11 and a cover 12 connected to the main body 11 to form the receiving chamber 3, and the cover 12 (or 12a) is a transparent structure. Users can directly observe the storage status of at least two hand warmers 2 inside without opening the cover, or directly observe the indicator unit 30a (such as a display screen) set on the compartment 10 for displaying status information through the transparent cover.

In addition, the hand warmer assembly of the present invention can also have another structural manifestation. In other embodiments, a portable hand warmer assembly is provided, including at least two hand warmers 2 and a portable storage box 200 (such as the portable hand warmer charging device 10d shown in the aforementioned FIGS. 30-31). The storage box 200 includes a case body 10 and a cover body 12 that can cover the storage body. The case body 10 has a receiving portion 3a for placing at least two hand warmers 2. The storage box does not require a deep groove shaped cavity, but has a placement portion 3a (such as a flat placement portion) for placing at least two hand warmers 2, and then multiple hand warmer components 2 are stably stored in the placement portion 3a of the case body 10 through the cover body 12. When the portable storage box is used as a charging device, the charging device is equipped with a first charging unit 131 (or wireless charging coil 3c) corresponding to the placement portion 3a, and at least two of the hand warmers 2 are equipped with a second charging unit 24; at least two of the hand warmers 2 are placed in the placement portion 3a, and the first charging unit 131 can charge the second charging unit 24 through contact charging or wireless charging.

At the same time, the above-mentioned case body 10 with charging function can also be equipped with a first charging interface (i.e. external charging interface 10a) that is electrically connected to the first circuit board 13, for charging other electrical appliances except for the hand warmer unit (i.e. external discharge); and the hand warmer 2 can also be independently equipped with a second charging interface 25 for external power cords.

Furthermore, the second circuit board 23 inside the hand warmer 2 is integrated with a charge and discharge management module, which includes a flash charging circuit that complies with flash charging protocols (such as PD flash charging protocol, QC flash charging protocol, etc.). The second charging interface 25 (preferably using a Type-C interface that supports bidirectional charging and discharging) provided on the hand warmer unit 2 is electrically connected to the flash charging circuit on the second circuit board 23. When in use, the second charging interface 25 has bidirectional power transmission characteristics: on one hand, when an external power source is connected, the input power is quickly charged to the second battery 21 through a flash charging circuit; on the other hand, when users need to charge other external electronic devices such as mobile phones and tablets, the second circuit board 23 can call the flash charging circuit to output the stored electrical energy of the second battery 21 in flash charging mode through the second charging interface 25. Through the coordination of the above hardware circuits and interfaces, when carrying the hand warmer unit 2 alone outside, it is not only a heating device, but also a portable power bank that meets modern flash charging standards, greatly enriching the functionality of the product and enhancing the user's emergency experience.

Furthermore, the hand warmer unit 2 is equipped with a power switch 210, which is used to control the on and off of the heating element 22 and adjust different heating gears. For example, by short pressing the power switch, users can switch between high, medium, and low temperatures to meet their heating needs in different environmental temperatures; Long press the power switch for 3 seconds to activate or deactivate the hand warmer unit 2.

The portable hand warmer storage box or portable hand warmer component described in this embodiment has other specific physical and mechanical structural features related to the case body 10, receiving chamber 3, and cover 12, such as the specific orientation of the opening 101 (upward or sideways), the position and orientation of the indicator unit 30a (such as a display screen), the specific placement method of the hand warmer unit 2 (such as flat placement or vertical insertion with end face 2b, partial exposure of upper surface 2a, etc.), as well as the cooperation structure between the groove 25 on the hand warmer unit 2 and the embedded block 1121 in the receiving chamber 3, and the charging compartment is equipped with a display screen and the hand warmer unit can also be equipped with a display screen, all of which are consistent with the aforementioned specific embodiments. The structure, position, and coordination relationship detailed in the accompanying drawings (such as FIGS. 16 to 27) are completely consistent and will not be repeated here. Technicians in this field should understand that all mechanical form designs, placement cavity arrangements, and storage posture features of the hand warmer unit related to the charging compartment (or charging device) in the aforementioned embodiments can be equivalently and seamlessly applied to the portable storage box for physical storage only described in this embodiment, in order to jointly achieve the beneficial effects of convenient storage, carrying, and status display of the present invention.

It can be understood that the above case body 10 can have only one receiving chamber 3 /receiving portion 3a (see FIG. 31), and the number of the hand warmer 2 can be one or more, such as three hand warmers 2, the one or more hand warmers 2 can be received in the only one receiving chamber 3; or the number of the receiving chambers 3/receiving portions 3a can correspond to the number of the hand warmers 2.

In present application, the hand warmers 2 are electronic hand warmers 2, and the upper surface 2a is the warming surface, it can be understood that other surfaces of each of the hand warmers 2 can also be warming surfaces. The surfaces of the hand warmers 2 are frosted surfaces with a certain hardness, and it is not flexible. The entire electronic hand warmers 2 can have a small size, substantially be less or equal to a hand of human, for example a length of 5-10cm, a width of 3-5cm; and a height of 1-3cm.

In other embodiments, see FIG. 34, the storage box 200 has a handle 200a for facilitating carrying the storage box, and chain or rope can replace the handle 200a.

It should be noted that all directional indications (such as up, down, left, right, front, back...) in the embodiments of the present disclosure are only used to explain a relative positional relationship between components, motion situations, etc. at a certain specific attitude (as shown in the figures). If the specific attitude changes, the directional indication also correspondingly changes.

In addition, the descriptions of "first", "second", etc. in the present disclosure are only used for descriptive purposes, and cannot be understood as indicating or implying its relative importance or implicitly indicating the number of technical features indicated. Therefore, features defined by "first" and "second" can explicitly instruct or impliedly include at least one feature. In addition, "and/or" in the entire text includes three solutions. A and/or B is taken as an example, including technical solution A, technical solution B, and technical solutions that both A and B satisfy. In addition, the technical solutions between the various embodiments can be combined with each other, but it needs be based on what can be achieved by those of ordinary skill in the art. When the combination of the technical solutions is contradictory or cannot be achieved, it should be considered that such a combination of the technical solutions does not exist, and is not within the scope of protection claimed by the present disclosure.

The above descriptions are only preferred embodiments of the present disclosure, and are not intended to limit the patent scope of the present disclosure. Any equivalent structural transformation made by using the content of the specification and the drawings of the present disclosure under the invention idea of the present disclosure, directly or indirectly applied to other related technical fields, shall all be included in the scope of patent protection of the present disclosure.

What is claimed is:

1. A portable hand warmer assembly, comprising: two electronic hand warmers, wherein each of the electronic hand warmers is provided with a heating element and comprises an upper surface for heating and a lower surface opposite to the upper surface; wherein the electronic hand warmers are capable of being detachably connected to each other in a manner that each of the electronic hand warmers is capable of warming individually through the upper surface, or the two electronic hand warmers are stacked by stacking the lower surfaces together to form a combined unit for warming; and a storage box, comprising a case body with at least one receiving chamber for receiving the two electronic hand warmers, wherein the case body comprises an opening corresponding to the at least one receiving chamber, allowing the two electronic hand warmers to be inserted into the at least one receiving chamber through the opening individually or in the combined unit.

2. The portable hand warmer assembly according to claim 1, wherein the case body further comprises a first circuit board, a first charging unit, and a first battery, and the first battery and the first charging unit are electrically connected to the first circuit board; each of the electronic hand warmers is further provided with a second circuit board, a second battery, and a second charging unit, and the heating element, the second battery, and the second charging unit are all electrically connected to the second circuit board; after one of the electronic hand warmers is received in the at least one receiving chamber, the second charging unit is close to or in contact with the first charging unit; the first charging unit is configured to output the electrical energy, which is output by the first circuit board, to the second charging unit; and the second charging unit charges, via the second circuit board, the second battery with the electrical energy output by the first charging unit, and each of the electronic hand warmers is capable of warming hand through working of the heating element.

3. The portable hand warmer assembly according to claim 2, wherein the first charging unit is a conductive probe, and the conductive probe is arranged at a bottom of the at least one receiving chamber; the second charging unit is a charging contact; the charging contact is located at a bottom of each of the electronic hand warmers; and the charging contact is in contact with the conductive probe when one of the electronic hand warmers is received in the at least one receiving chamber.

4. The portable hand warmer assembly according to claim 1, wherein the at least one receiving chamber comprises two receiving chambers, and the two electronic hand warmers are detached from each other and received in the two receiving chambers respectively; the case body is equipped with a charging unit, and the two electronic hand warmers can be charged when received in the receiving chambers.

5. The portable hand warmer assembly according to claim 1, wherein the lower surfaces are detachably connected through one of mutual magnetic attraction, snap fasteners, or Velcro.

6. The portable hand warmer assembly according to claim 2, wherein the case body is equipped with a first charging interface for charging other electrical appliances besides the electronic hand warmers, and the first charging interface is electrically connected to the first circuit board.

7. The portable hand warmer assembly according to claim 2, wherein each of the electronic hand warmers is equipped with a second charging interface, which is electrically connected to the second circuit board; and the second charging interface is used for external power supply to charge the second battery; the second circuit board is further equipped with a flash charging circuit, which is used to control a power of the second battery to be output in a flash charging mode through the second charging interface to achieve flash charging external devices.

8. A portable hand warmer assembly, comprising: at least two electronic hand warmers; and a storage box, comprising a case body with at least one receiving chamber for receiving the at least two electronic hand warmers, and an openable cover covering the case body.

9. The portable hand warmer assembly according to claim 8, wherein the storage box is a charging device, which is equipped with a first charging unit corresponding to the at least one receiving chamber; each of the at least two electronic hand warmers is equipped with a second charging unit; after one of at least two electronic hand warmers is received in the at least one receiving chamber, and the first charging unit charges the second charging unit.

10. The portable hand warmer assembly according to claim 8, wherein the first charging unit charges the second charging unit through contact or through wireless manner.

11. A storage box, comprising: a case body with at least one receiving portion for receiving at least one electronic hand warmer, and the case body comprises a first charging unit, after the at least one electronic hand warmer is received in the at least one receiving portion, the at least one electronic hand warmer is charged by the first charging unit.

12. The storage box according to claim 11, wherein the case body is provided with an indicator unit configured to indicate a state of the case body and/or a state of the at least one electronic hand warmer.

13. The storage box according to claim 11, wherein the first charging unit comprises a first circuit board, a first charging unit, and a first battery; the first battery and the first charging unit are electrically connected to the first circuit board; when the at least one electronic hand warmer is received in the at least one receiving portion, the first circuit board is used to output electrical energy output from the first battery from the first charging unit; the first charging unit is designed to be compatible with a second charging unit on the at least one electronic hand warmer, and transfer electrical energy output from the first circuit board to the second charging unit on the at least one electronic hand warmer.

14. The storage box according to claim 13, wherein the first charging unit is a conductive probe, and the conductive probe is arranged at a bottom of the at least one receiving portion; the first circuit board is located at the bottom of the at least one receiving portion, and the first battery is located behind the at least one receiving portion.

15. The storage box according to claim 11, wherein the case body comprises an opening facing upwards, the at least one electronic hand warmer is placed in the at least one receiving portion through the opening;
the case body further comprises an indicator unit configured to indicate a state of the case body and/or a state of the at least one electronic hand warmer.

16. The storage box according to claim 11, wherein the case body comprises an opening facing upwards, the at least one electronic hand warmer is placed in the at least one receiving portion through the opening; wherein the at least one electronic hand warmer comprises an upper surface and an end surface perpendicular to the upper surface, the receiving portion is concave and is configured for the at least one electronic hand warmer being vertically inserted into the receiving portion, after the at least one electronic hand warmer is placed in the at least one receiving portion, the end surface of the electronic hand warmer faces upwards, and a part of the upper surface is exposed at the opening.

17. The storage box according to claim 11, wherein the at least one receiving portion comprises two separated receiving chambers, the case body comprises a side opening, the at least one electronic hand warmer comprises two electronic hand warms, and the two electronic hand warms are inserted in the receiving chambers through the opening; after the electronic hand warmers are inserted in the receiving chambers, a part of each of the electronic hand warmers is exposed at the side opening.

18. The storage box according to claim 11, wherein the at least one receiving portion comprises two separated receiving chambers, the receiving chambers are arranged in parallel, the case body comprises two openings corresponding to the receiving chambers.

19. The storage box according to claim 12, wherein the indicator unit is a display screen.

20. The storage box according to claim 11, wherein the storage box further comprises an openable cover covering the at least one receiving portion, and the cover is transparent to allow the at least one electronic hand warmer received in at least one receiving portion to be visible.
